SEQUENCE LISTING (42 sequences)

SEQ ID NO: 1 - DNA, 51 bp, Artificial Sequence
  Synthetic
ggggacaacttttctatacaaagttgccatggcttcgacgtcggccaccga

SEQ ID NO: 2 - DNA, 46 bp, Artificial Sequence
  Synthetic
ggggacaactttattatacaaagttgtgatccggaagctttcctcc

SEQ ID NO: 3 - DNA, 20 bp, Artificial Sequence
  Synthetic
tttgggttcgcacagttgac

SEQ ID NO: 4 - DNA, 21 bp, Artificial Sequence
  Synthetic
aagtctttctcccgaggaagc

SEQ ID NO: 5 - DNA, 20 bp, Artificial Sequence
  Synthetic
agcatcgatccgacactcac

SEQ ID NO: 6 - DNA, 20 bp, Artificial Sequence
  Synthetic
tgttcagccgaatcttgtcc

SEQ ID NO: 7 - DNA, 19 bp, Artificial Sequence
  Synthetic
aggtcccaggatccagtcc

SEQ ID NO: 8 - DNA, 20 bp, Artificial Sequence
  Synthetic
tgttcagccgaatcttgtcc

SEQ ID NO: 9 - DNA, 22 bp, Artificial Sequence
  Synthetic
cactcagggaatacacgcctac

SEQ ID NO: 10 - DNA, 23 bp, Artificial Sequence
  Synthetic
atcttggtcttagggtcttcagg

SEQ ID NO: 11 - DNA, 18 bp, Artificial Sequence
  Synthetic
agcggattccaggtgagg

SEQ ID NO: 12 - DNA, 20 bp, Artificial Sequence
  Synthetic
ttgttcaggcggatcttgtc

SEQ ID NO: 13 - DNA, 22 bp, Artificial Sequence
  Synthetic
gtcatcaccatcgccaataacg

SEQ ID NO: 14 - DNA, 22 bp, Artificial Sequence
  Synthetic
tgtggtgaagtggtagtccatg

SEQ ID NO: 15 - DNA, 21 bp, Artificial Sequence
  Synthetic
tgagaagtgggtggactttgc

SEQ ID NO: 16 - DNA, 20 bp, Artificial Sequence
  Synthetic
atggcattcttgagcagctc

SEQ ID NO: 17 - DNA, 20 bp, Artificial Sequence
  Synthetic
ttccttcaagtgctgtgcag

SEQ ID NO: 18 - DNA, 21 bp, Artificial Sequence
  Synthetic
gcagatcgagtcacagcaatg

SEQ ID NO: 19 - DNA, 20 bp, Artificial Sequence
  Synthetic
caagcaggcacacacaatgg

SEQ ID NO: 20 - DNA, 22 bp, Artificial Sequence
  Synthetic
agtgtttgttcctcggagtgag

SEQ ID NO: 21 - DNA, 22 bp, Artificial Sequence
  Synthetic
ttcaagtatgcccgggttactc

SEQ ID NO: 22 - DNA, 20 bp, Artificial Sequence
  Synthetic
ttcctcgacgtttgatcagc

SEQ ID NO: 23 - protein, 15 aa, Artificial Sequence
  Synthetic
DDSSAYRSVDEVNYW

SEQ ID NO: 24 - protein, 15 aa, Artificial Sequence
  Synthetic
KLEEKQKSDAEEDGG

SEQ ID NO: 25 - protein, 15 aa, Artificial Sequence
  Synthetic
DAEEDGGTGSQDEED

SEQ ID NO: 26 - protein, 15 aa, Artificial Sequence
  Synthetic
GKLLRSQSQASLTGL

SEQ ID NO: 27 - protein, 15 aa, Artificial Sequence
  Synthetic
RFIIGSVSEDNSEDE

SEQ ID NO: 28 - protein, 15 aa, Artificial Sequence
  Synthetic
GSVSEDNSEDEISNL

SEQ ID NO: 29 - protein, 15 aa, Artificial Sequence
  Synthetic
FSATVRASQGPVYKG

SEQ ID NO: 30 - protein, 15 aa, Artificial Sequence
  Synthetic
PAPSRTASFSESRAD

SEQ ID NO: 31 - protein, 14 aa, Artificial Sequence
  Synthetic
HYHCAEGSQEECDK

SEQ ID NO: 32 - protein, 15 aa, Artificial Sequence
  Synthetic
KEFRRTRSLHGPCPV

SEQ ID NO: 33 - protein, 15 aa, Artificial Sequence
  Synthetic
GEEPTVYSDDEEPKD

SEQ ID NO: 34 - protein, 15 aa, Artificial Sequence
  Synthetic
FHMRSSMSGLHLVKQ

SEQ ID NO: 35 - protein, 15 aa, Artificial Sequence
  Synthetic
TYRIGHHSTSDDSSA

SEQ ID NO: 36 - protein, 15 aa, Artificial Sequence
  Synthetic
AKRRRLSSLRASTSK

SEQ ID NO: 37 - protein, 15 aa, Artificial Sequence
  Synthetic
RLSSLRASTSKSESS

SEQ ID NO: 38 - protein, 15 aa, Artificial Sequence
  Synthetic
NGPQRSLSLSLEKEM

SEQ ID NO: 39 - protein, 15 aa, Artificial Sequence
  Synthetic
RDSGRGDSVSDNGSE

SEQ ID NO: 40 - protein, 15 aa, Artificial Sequence
  Synthetic
PAPSRTASFSESRAD

SEQ ID NO: 41 - protein, 15 aa, Artificial Sequence
  Synthetic
IAKRRRLSSLRASTS

SEQ ID NO: 42 - protein, 15 aa, Artificial Sequence
  Synthetic
TYRIGHHSTSDDSSA

(19) United States
(12) Patent Application Publication
(10) Pub. No.: US 2021/0267939 A1
(43) Pub. Date: Sep. 2, 2021

(54) COMPOSITIONS AND METHODS FOR TREATING NAFLD/NASH AND RELATED DISEASE PHENOTYPES

(71) Applicant: Duke University, Durham, NC (US)

(72) Inventors: Christopher B. NEWGARD, Durham, NC (US); Phillip WHITE, Durham, NC (US); Thomas GRENIER-LAROUCHE, Durham, NC (US)

(21) Appl. No.: 17/254,119

(22) PCT Filed: Jun. 18, 2019
(86) PCT No.: PCT/US2019/037739

§ 371 Date: Dec. 18, 2020

Related U.S. Application Data
(60) Provisional application No. 62/686,154, filed on Jun. 18, 2018.

(51) Int. Cl. A61K 31/381

(52) U.S. Cl. CPC A61K 31/381

(57) ABSTRACT

The present invention relates to compositions and methods for the treatment of NAFLD. Specifically, the present invention relates to compositions comprising one or more BCDKH agonists and methods of using the same for the treatment of NAFLD.

### PRIORITY

This application claims priority to U.S. Provisional Application No. 62/686,154, filed Jun. 18, 2018, the entire contents of which are incorporated herein by reference.

### FEDERAL FUNDING

This invention was made with government support under Federal Grant Nos. PO1-DK58398, PO1-DK100425, PO1-DK083439, PO1-DK62306, PO1-DK92921 and K08HL135275, awarded by the NIH. The government has certain rights in the invention.

### TECHNICAL FIELD

The present invention relates to compositions and methods for treating nonalcoholic fatty liver disease and related disease phenotypes. Specifically, invention relates to compositions comprising one or more branched-chain ketoacid dehydrogenase complex (BCKDH) agonists and methods of using the same for treatment of NAFLD.

### BACKGROUND

Non-alcoholic fatty liver disease (NAFLD) is characterized by neutral lipid accumulation in the liver. NAFLD encompasses a histologic spectrum ranging from isolated hepatic steatosis to nonalcoholic steatohepatitis (NASH) characterized by lipid accumulation, inflammation, hepatocyte ballooning, and varying degrees of fibrosis. This more pathogenic form of NAFLD progresses to fibrosis in approximately 35% of patients, significantly raising the risk for development of hepatocellular carcinoma (HCC), cirrhosis, and acute liver failure. Advanced NAFLD is also a significant risk factor for development of type 2 diabetes and cardiovascular diseases (CVD). The severity of hepatic fibrosis is the primary predictor of increased morbidity and mortality in patients with NAFLD.

The prevalence of nonalcoholic fatty liver disease (NAFLD) continues to increase with the growing obesity epidemic. The obesity pandemic has driven a sharp increase in the incidence of NAFLD in recent years to an estimated incidence in the United States of 25%. NALFD-related liver failure is now comparable to hepatitis C as a primary cause of liver transplants in the United States. Coincidentally, the rising tide of NAFLD has also lowered the quality of the available liver donor pool. Accordingly, effective methods for treating NAFLD are needed.

### SUMMARY

In some embodiments, provided herein are methods for treating metabolic disease in a subject. The methods include administering to the subject a therapeutically effective amount of one or more branched-chain ketoacid dehydrogenase complex (BCKDH) agonists.

In some embodiments, provided herein are compositions comprising one or more branched-chain ketoacid dehydrogenase complex (BCKDH) agonists for use in a method of treating metabolic disease in a subject.

In accordance with any of the embodiments described herein, the one or more BCDHK agonists may be selected from BDK kinase inhibitors, PPM1K agonists, and combinations thereof. In some embodiments, the one or more BCDHK agonists comprise one or more benzothiophene carboxylate derivatives. In some embodiments, the one or more benzothiophene carboxylate derivatives are selected from (S)-α-cholorophenylproprionate ((S)-CPP)), (N-(4-amino-1,2,5-oxadiazol-3-yl)-3,6-dichlorobenzo[b]thiophene-2-carboxamide) (BT1), (3,6-dichlorobenzo[b]thiophene-2-carboxylic acid) (BT2), (3-chloro-6-fluorobenzo[b]thiophene-2-carboxylic acid) (BT2F), and (N-(4-acetamido-1,2,5-oxadiazol-3-yl)-3,6-dichlorobenzo[b]thiophene-2-carboxamide) (BT3).

In accordance with any of the embodiments described herein, the metabolic disease may be obesity, insulin-resistance, diabetes, metabolic syndrome, alcoholic steatohepatitis, or NAFLD.

In one aspect, provided herein are methods for treating NAFLD in a subject, comprising administering to the subject a therapeutically effective amount of (3,6-dichlorobenzo[b]thiophene-2-carboxylic acid) (BT2).

### BRIEF DESCRIPTION OF THE DRAWINGS

FIG. 1. Metabolic effects of BT2 treatment or PPM1K expression in Zucker fatty rats. (A) BCKDH activity in liver, heart and skeletal muscle (Skm) tissue of BT2 (20 mg/kg i.p.) or vehicle (Veh)-treated Zucker fatty rats (ZFR). (B) Representative immunoblots of total and phospho-ser 293 of BCKDH e1a. Effects of BT2 on circulating branched chain amino acids (BCAA) (C) and branched chain keto acids (BCKA) (D). Body (E) and tissue (F) weights measured at the end of the study period. (G) Liver triacylglyceride content in BT2- and Veh-treated ZFR. Glucose (H) and insulin (I) excursions during a 1 g/kg IP glucose tolerance test. Data in panels A-I are expressed as the mean±SEM, n=8-10 animals per group. * P<0.05, ** P<0.01, *** P<0.001. Recombinant adenoviruses expressing human PPM1K (Ad-CMV-PPM1K) or GFP (Ad-CMV-GFP) were administered to 14 week-old Zucker fatty rats (ZFR) via tail vein. (J) Expression of human and endogenous (rat) PPM1K mRNA in liver. (K) Effect of each adenovirus on BCKDH activity in liver and heart tissue. (L) Representative immunoblots of total and phospho-ser 293 of BCKDH e1a, PPM1K, and GFP in liver. Effects of each adenovirus on circulating BCAA (M) and BCKA (N). Body (O) and tissue (P) weights measured at the end of the study period. (Q) Liver triacylglyceride content. Glucose (R) and insulin (S) excursions during a 1 g/kg i.p. glucose tolerance test. Data in panels J-S are expressed as the mean±SEM, n=6-10 animals per group. * P<0.05, ** P<0.01, *** P<0.001. See FIGS. 6 and 7 for related information.

FIG. 2. Phospho-proteomics reveals additional targets of BDK and PPM1K in liver. (A) Study workflow. Panels (B) and (C) show flanking amino acid sequences of all phosphosites downregulated by BT2 or Ad-CMV-PPM1K treatments, respectively. Thresholds of ≥−0.585 Log 2 fold change in phosphorylation and statistical significance of P<0.05 were used (n=3 samples per group). The modulated serine in each phosphoprotein is highlighted in red. Consensus phosphosite motif sequences generated for BT2 (D) and Ad-CMV-PPM1K (E) modulated phosphosites. (F) Representative immunoblot for phospho-ser454 and total ATP citrate lyase (ACL), BDK, and GAPDH proteins in liver tissues from BT2- or Veh-treated Zucker fatty rats (ZFR). (G) Representative immunoblot for phospho-ser454 and total ATP citrate lyase (ACL), PPM1K, and GAPDH in liver tissues from Ad-CMV-PPM1K- or Ad-CMV-GFP-treated ZFR. Representative immunoblots in panels F and G are shown alongside densitometric analyses of pACL/total ACL. Data are expressed as mean±SEM from n=5 animals per group. ** P<0.01.

FIG. 3. Subcellular localization of ACL, BDK and PPM1K and effect of BDK overexpression on ACL phosphorylation in vitro. (A) Representative immunoblots of ACL, BDK, PPM1K, the mitochondrial markers ETFA and COXIV, and the cytosolic marker GAPDH in cytosolic and mitochondrial fractions of liver from lean Wistar rats sacrificed in the ad-libitum fed or overnight fasted states. (B-C) Volcano plots showing the subcellular location of proteins containing phosphopeptides found to be downregulated by BT2 or Ad-CMV-PPM1K treatments, respectively. (D) Effect of Ad-CMV-BDK overexpression in Fao cells on ACL phosphorylation on ser454, total ACL, BCKDH e1a phosphorylation on ser293, total e1a, and BDK protein abundance. Densitometric analysis of pACL/ACL ratio is shown below the representative blot. Data are mean±SEM representing n=3 independent experiments. ** P<0.01. (E) Confocal images of Hek293 cells transfected with plasmid encoding a GFP tagged BDK lacking the mitochondrial targeting sequence, under control of a CMV promoter (CMV-ΔMTS-BDK-GFP) or CMV-GFP control constructs co-stained with MitoTracker (red) and Hoechst (blue). (F) Effect of Ad-CMV-ΔMTS-BDK overexpression in Fao cells on ACL phosphorylation on ser454, total ACL, BCKDH e1a phosphorylation on ser293, total e1a, and BDK protein abundance. Densitometric analysis of pACL/ACL ratio is shown below the representative blot, as mean±SEM of 3 independent experiments. ** P<0.01. (G) Studies with purified ACL, protein kinase A (PKA), and BCKDH subunit proteins. The lower panel demonstrates direct phosphorylation of ACL and the e1a subunit of BCKDH by both BDK and protein kinase A (PKA). The Coomassie stain of the same gel is shown in the upper panel. See FIG. 8 for related experiments.

FIG. 4. BDK phosphorylates ACL and activates de novo lipogenesis in vivo. (A) Representative immunoblot of phospho-454 and total ATP citrate lyase (ACL) in unfractionated liver samples from the same fasted or fed Wistar rats used for the fractionation study shown in FIG. 3A. (B) ACL phosphorylation on ser454, total ACL, and BDK protein abundance in liver of Ad-CMV-BDK or Ad-CMV-βGAL-treated Wistar rats. (C) Densitometric analysis of pACL/ACL ratio. (D) Effect of Ad-CMV-BDK on rates of de novo lipogenesis (DNL) measured as incorporation of D20 into newly synthesized palmitate in liver. (E) D20 enrichment in plasma of Ad-CMV-BDK and Ad-CMV-βGAL-treated rats at sacrifice. (F) Body weights in Ad-CMV-BDK and Ad-CMV-βGAL-injected rats. Data in panels C-F are expressed as mean±SEM, n=4-6 rats per group. ** P<0.01. (G) Dual localization of BDK and PPM1K in the cytosolic and mitochondrial subcellular compartments enables these enzymes to simultaneously modify the phosphorylation states of ACL and BCKDH, resulting in coordinated regulation of lipid and BCAA metabolism.

FIG. 5. Transcriptional regulation of BDK and PPM1K by ChREBP. (A) Conservation across mammalian species of an enhancer containing a ChREBP binding site proximal to the human BDK gene. The red arrow locates the ChREBP binding site at a multicolored H3K27Ac “peak” which is indicative of an active regulatory element. Vertical black hatch marks to the right of each mammal indicates conserved sequence relative to the human genome. Note the absence of the element in mice, and its retention in rats. (B) ChREBP-β mRNA expression is positively correlated with BDK mRNA expression in liver biopsies taken from 86 overnight fasted human subjects with non-alcoholic fatty liver disease (NAFLD). (C) Effects of 4 hours of refeeding of high fructose (60% fructose) or standard chow diets to overnight fasted Wistar rats on hepatic transcript levels of known ChREBP response genes, as well as BDK and PPM1K. Data are mean±SEM, n=5 rats per group. ** P<0.01, *** P<0.001, **** P<0.0001, ***** P<0.00001, ****** P<0.000001. (D) Mouse (m) ChREBP-β and rat (r) Bckdk (BDK), and PPM1K mRNA expression in liver of Ad-CMV-mChREBP-β or Ad-CMV-GFP-treated Wistar rats. Data are mean±SEM, n=6-8 rats per group. ** P<0.01, *** P<0.001. (E) Schematic summary showing that fructose feeding activates ChREBP-β to drive transcription of the lipogenic program (component genes shown in burgundy), now including BDK as a post-translational activator of the pathway. ChREBP-β induction also leads to repression of PPM1K expression.

FIG. 6. Effect of BT2 on RER, plasma lactate, hepatic acylcarnitines, and plasma lipids. Related to FIG. 1. Fourteen week-old Zucker fatty rats (ZFR) were treated with the BDK inhibitor BT2 (20 mg/kg IP) or vehicle (Veh) daily for one week. (A) A cohort of ZFR were placed in metabolic cages at 9 am on day 6 immediately following Veh or BT2 administration and VO2, heat production, and the respiratory exchange ratio (RER) were monitored for the ensuing 7 hours. (B) Concentrations of plasma lactate (LACT). (C) Hepatic acylcarnitine levels. (D) Concentrations of plasma triacylglyceride (TG), cholesterol, glycerol, non-esterified fatty acids (NEFA), hydroxybutyrate (HB) and ketones (KET). All data are expressed as the mean±SEM, n=8-10 animals per group. * P<0.001.

FIG. 7. Effect of adenovirus-mediated PPM1K overexpression on plasma lactate, hepatic acyl-carnitines, and plasma lipids. Related to FIG. 1. Recombinant adenoviruses expressing human PPM1K or GFP (control) were administered to 14 week-old Zucker fatty rats (ZFR) via tail vein. (A) Concentrations of plasma lactate (LACT). (B) Hepatic acylcarnitine levels. (C) Concentrations of plasma triacylglyceride (TG), cholesterol, glycerol, non-esterified fatty acids (NEFA), hydroxybutyrate (HB) and ketones (KET). All data are expressed as the mean±SEM, n=6-10 animals per group. * P<0.01.

FIG. 8. Regulation of ACL by BDK is independent of AKT activity. Related to FIG. 3. Fao hepatoma cells were transfected with recombinant adenoviruses expressing human BDK or β gal (control) for 72 hours. Cells were exposed to the pan Akt inhibitor A6730 or vehicle control for 1 hour prior to lysis. Immunoblots for pACL ser454, total ACL, pAKT ser473, total AKT, and BDK are shown in panel (A). Antibodies for pAKT and total AKT react with AKT1/2/3. Western blots for pAKT ser473 and total AKT from liver of vehicle or BT2 treated ZFR and Ad-CMV-PPM1K or AD-CMV-GFP treated ZFR are shown in panel (B). Fao cells were transfected with recombinant adenoviruses expressing V5 tagged human BDK, β gal or ACL. proteins were purified by immunoprecipitation with an anti-V5 column and ACL was co-incubated with purified β gal (control) or BDK in the presence of ATP. Panel (C) shows effect of incubation with purified β gal (control) or BDK on ACL phosphorylation on ser455

### DETAILED DESCRIPTION

The propensity of an individual to develop NAFLD is dictated by a combination of genetics, lifestyle, diet, and insulin sensitivity. Hepatic triglyceride pools are influenced by supply of adipose derived non-esterified fatty acids (NEFA) to the liver, hepatic de novo lipogenesis (DNL), NEFA export in very low-density lipoprotein (VLDL), and hepatic rates of beta oxidation and ketogenesis. Metabolic flux indicate that high hepatic fat content is associated with three-fold higher rates of DNL but no difference in adipose efflux of NEFA or production of VLDL. Thus, hepatic DNL appears to be a distinguishing feature of NAFLD. Furthermore, beta oxidation to the TCA cycle rather than ketogenesis may also be an underlying feature of persons with NAFLD.

The present disclosure is predicated, at least in part, on the discovery that hepatic DNL, a distinguishing feature of NAFLD, may be regulated in part by the levels of the branched chain α-keto acid dehydrogenase kinase (BDK) and phosphatase (PPM1K) in the subject.

For the purposes of promoting an understanding of the principles of the present disclosure, reference will now be made to preferred embodiments and specific language will be used to describe the same. It will nevertheless be understood that no limitation of the scope of the disclosure is thereby intended, such alteration and further modifications of the disclosure as illustrated herein, being contemplated as would normally occur to one skilled in the art to which the disclosure relates.

### Definitions

Unless otherwise defined, all technical terms used herein have the same meaning as commonly understood by one of ordinary skill in the art to which this disclosure belongs. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

The use of the terms “a” and “an” and “the” and “at least one” and similar referents in the context of describing the invention (especially in the context of the following claims) are to be construed to cover both the singular and the plural, unless otherwise indicated herein or clearly contradicted by context.

The use of the term “at least one” followed by a list of one or more items (for example, “at least one of A and B”) is to be construed to mean one item selected from the listed items (A or B) or any combination of two or more of the listed items (A and B), unless otherwise indicated herein or clearly contradicted by context.

As used herein, the term “about” is used to provide flexibility to a numerical range endpoint by providing that a given value may be “slightly above” or “slightly below” the endpoint without affecting the desired result. In some embodiments, “about” may refer to variations of in some embodiments ±20%, in some embodiments ±10%, in some embodiments ±5%, in some embodiments ±1%, in some embodiments ±0.5%, and in some embodiments ±0.1% from the specified amount.

As used herein, the terms “comprise”, “include”, and linguistic variations thereof denote the presence of recited feature(s), element(s), method step(s), etc. without the exclusion of the presence of additional feature(s), element(s), method step(s), etc.

Recitation of ranges of values herein are merely intended to serve as a shorthand method of referring individually to each separate value falling within the range, unless otherwise-indicated herein, and each separate value is incorporated into the specification as if it were individually recited herein. For example, if a concentration range is stated as 1% to 50%, it is intended that values such as 2% to 40%, 10% to 30%, or 1% to 3%, etc., are expressly enumerated in this specification. These are only examples of what is specifically intended, and all possible combinations of numerical values between and including the lowest value and the highest value enumerated are to be considered to be expressly stated in this disclosure.

The term “amino acid” refers to natural amino acids, unnatural amino acids, and amino acid analogs, all in their D and L stereoisomers, unless otherwise indicated, if their structures allow such stereoisomeric forms.

Natural amino acids include alanine (Ala or A), arginine (Arg or R), asparagine (Asn or N), aspartic acid (Asp or D), cysteine (Cys or C), glutamine (Gln or Q), glutamic acid (Glu or E), glycine (Gly or G), histidine (His or H), isoleucine (Ile or I), leucine (Leu or L), Lysine (Lys or K), methionine (Met or M), phenylalanine (Phe or F), proline (Pro or P), serine (Ser or S), threonine (Thr or T), tryptophan (Trp or W), tyrosine (Tyr or Y) and valine (Val or V).

Unnatural amino acids include, but are not limited to, azetidinecarboxylic acid, 2-aminoadipic acid, 3-aminoadipic acid, beta-alanine, naphthylalanine (“naph”), aminopropionic acid, 2-aminobutyric acid, 4-aminobutyric acid, 6-aminocaproic acid, 2-aminoheptanoic acid, 2-aminoisobutyric acid, 3-aminoisbutyric acid, 2-aminopimelic acid, tertiary-butylglycine (“tBuG”), 2,4-diaminoisobutyric acid, desmosine, 2,2′-diaminopimelic acid, 2,3-diaminopropionic acid, N-ethylglycine, N-ethylasparagine, homoproline (“hPro” or “homoP”), hydroxylysine, allo-hydroxylysine, 3-hydroxyproline (“3Hyp”), 4-hydroxyproline (“4Hyp”), isodesmosine, allo-isoleucine, N-methylalanine (“MeAla” or “Nime”), N-alkylglycine (“NAG”) including N-methylglycine, N-methylisoleucine, N-alkylpentylglycine (“NAPG”) including N-methylpentylglycine. N-methylvaline, naphthylalanine, norvaline (“Norval”), norleucine (“Norleu”), octylglycine (“OctG”), ornithine (“Orn”), pentylglycine (“pG” or “PGly”), pipecolic acid, thioproline (“ThioP” or “tPro”), homoLysine (“hLys”), and homoArginine (“hArg”).

The term “amino acid analog” refers to a natural or unnatural amino acid where one or more of the C-terminal carboxy group, the N-terminal amino group and side-chain bioactive group has been chemically blocked, reversibly or irreversibly, or otherwise modified to another bioactive group. For example, aspartic acid-(beta-methyl ester) is an amino acid analog of aspartic acid; N-ethylglycine is an amino acid analog of glycine; or alanine carboxamide is an amino acid analog of alanine. Other amino acid analogs include methionine sulfoxide, methionine sulfone, S-(carboxymethyl)-cysteine, S-(carboxymethyl)-cysteine sulfoxide and S-(carboxymethyl)-cysteine sulfone.

As used herein, the term “biomarker” refers to a naturally occurring biological molecule present in a subject at varying concentrations useful in predicting the risk, incidence, or severity of a disease or a condition, such as NAFLD or other related disease phenotypes. For example, the biomarker can be a protein or any conventional metabolites that present in higher or lower amounts in a subject at risk for, or suffering from, NAFLD or related disease phenotypes. In some embodiments, the biomarker is a protein. A biomarker may also comprise any naturally or non-naturally occurring polymorphism (e.g., single-nucleotide polymorphism [SNP]) present in a subject that is useful in predicting the risk or incidence of NAFLD.

As used herein, the terms “co-administration” and variations thereof refer to the administration of at least two agent(s) or therapies to a subject. In some embodiments, the co-administration of two or more agents or therapies is concurrent. In other embodiments, a first agent/therapy is administered prior to a second agent/therapy. Those of skill in the art understand that the formulations and/or routes of administration of the various agents or therapies used may vary. The appropriate dosage for co-administration can be readily determined by one skilled in the art. In some embodiments, when agents or therapies are co-administered, the respective agents or therapies are administered at lower dosages than appropriate for their administration alone. Accordingly, co-administration may be especially desirable in embodiments where the co-administration of two or more agents results in sensitization of a subject to beneficial effects of one of the agents via co-administration of the other agent.

The term “carrier” as used herein refers to any pharmaceutically acceptable solvent of agents that will allow a therapeutic composition to be administered to the subject. A “carrier” as used herein, therefore, refers to such solvent as, but not limited to, water, saline, physiological saline, oil-water emulsions, gels, or any other solvent or combination of solvents and compounds known to one of skill in the art that is pharmaceutically and physiologically acceptable to the recipient human or animal.

As used herein, the terms “effective amount” or “therapeutically effective amount” are used interchangeably herein to refer to an amount sufficient to effect beneficial or desirable biological and/or clinical results.

As used herein, the term “fibrosis” refers to the formation of scar tissue in the liver. The term “fibrosis” may refer to “cirrhosis”, which is used herein to denote late-stage (e.g. advanced) fibrosis in the liver.

As used herein, the terms “non-alcoholic fatty liver disease” and “NAFLD” are used interchangeably to refer to a range of conditions affecting people who drink little to no alcohol characterized, at least in part, by excess fat stored in liver cells (e.g. steatosis). NAFLD may be characterized by any combination of features including steatosis, fibrosis, enlarged liver, fatigue, abdominal pain, abdominal swelling, enlarged blood vessels, enlarged breasts, enlarged spleen, red palms, and jaundice. NAFLD refers to a spectrum of conditions that may range in severity or degree, depending on the progression of the disease in a given individual. In some embodiments, non-alcoholic liver disease may refer to non-alcoholic steatohepatitis (“NASH”), a more severe form of NAFLD characterized by characterized by lipid accumulation, inflammation, hepatocyte ballooning, and varying degrees of fibrosis in the liver.

As used herein, the term “pharmaceutical composition” refers to the combination of an active agent with a carrier, inert or active, making the composition especially suitable for therapeutic use.

The term “pharmaceutically acceptable” as used herein refers to a compound or composition that will not impair the physiology of the recipient human or animal to the extent that the viability of the recipient is compromised. For example, “pharmaceutically acceptable” may refer to a compound or composition that does not substantially produce adverse reactions, e.g., toxic, allergic, or immunological reactions, when administered to a subject.

As used herein, the terms “prevent,” “prevention,” and preventing” may refer to reducing the likelihood of a particular condition or disease state (e.g., non-alcoholic steatohepatitis) from occurring in a subject not presently experiencing or afflicted with the condition or disease state. The terms do not necessarily indicate complete or absolute prevention. For example “preventing NASH” refers to reducing the likelihood of NASH occurring in a subject not presently experiencing or diagnosed with NASH. For example, preventing NASH may reduce the likelihood of NASH occurring in a subject currently diagnosed with mild NAFLD but not currently diagnosed with NASH. The terms may also refer to delaying the onset of a particular condition or disease state (e.g., NASH) in a subject not presently experiencing or afflicted with the condition or disease state. In order to “prevent” a condition, a composition or method need only reduce the likelihood and/or delay the onset of the condition, not completely block any possibility thereof “Prevention,” encompasses any administration or application of a therapeutic or technique to reduce the likelihood or delay the onset of a disease developing (e.g., in a mammal, including a human). Such a likelihood may be assessed for a population or for an individual.

The terms “sample” or “biological sample” as used interchangeably herein includes any suitable sample isolated from the subject. Suitable samples include, but are not limited to, a sample containing tissues, cells, and/or biological fluids isolated from a subject. Examples of samples include, but are not limited to, tissues, cells, biopsies, blood, lymph, serum, plasma, urine, saliva, mucus and tears. In one embodiment, the sample comprises a serum sample, a blood sample, or a plasma sample. A sample may be obtained directly from a subject or a control (e.g., by blood or tissue sampling) or from a third party (e.g., received from an intermediary, such as a healthcare provider or lab technician).

As used herein, the term “steatosis” refers to the accumulation of fat in the cells of the liver.

As used herein, the terms “subject” and “patient” are used interchangeably herein and refer to both human and nonhuman animals. The term “nonhuman animals” includes all vertebrates, e.g., mammals and non-mammals, such as nonhuman primates, sheep, dogs, cats, horses, cows, chickens, amphibians, reptiles, and the like. In some embodiments, the subject is a human. In some embodiments, the subject is a human. In particular embodiments, the subject may be overweight or obese. In particular embodiments, the subject may be male. In other embodiments, the subject may be female. In certain embodiments, the subject expresses the Ile148Met variant of PNPLA3. In certain embodiments, the subject is a human suffering from, or is at risk of suffering from, NAFLD or related disease phenotypes.

As used herein, “treatment,” “therapy” and/or “therapy regimen” refer to the clinical intervention made in response to a disease, disorder or physiological condition manifested by a patient or to which a patient may be susceptible. The aim of treatment includes the alleviation or prevention of symptoms, slowing or stopping the progression or worsening of a disease, disorder, or condition and/or the remission of the disease, disorder or condition. In some embodiments, treating NAFLD refers to the management and care of the subject for combating and reducing NAFLD. Treating NAFLD may reduce, inhibit, ameliorate and/or improve the onset of the symptoms or complications, alleviating the symptoms or complications of the disease, or eliminating the disease. As used herein, the term “treatment” is not necessarily meant to imply cure or complete abolition of the liver disease. Treatment may refer to the inhibiting or slowing of the progression of NAFLD or related disease phenotypes, reducing the incidence of NAFLD or related disease phenotypes, or preventing additional progression of NAFLD or related disease phenotypes. For example, treatment may refer to stopping the progression of NAFLD characterized by isolated steatosis to the more severe form of NAFLD, referred to herein as NASH.

### Compositions and Methods

In one aspect, disclosed herein are compositions and methods for treating metabolic disease in a subject. In some embodiments, the metabolic disease may be obesity, insulin-resistance, diabetes, metabolic syndrome, alcoholic steatohepatitis, NAFLD, or combinations thereof. For example, the metabolic disease may be NAFLD.

The methods for treating metabolic disease in a subject comprise administering to the subject a therapeutically effective amount of one or more therapeutic agents. For example, the one or more therapeutic agents may be one or more BCKDH agonists (e.g. activators of BCKDH). Suitable BCKDH agonists include small molecules, peptides, polypeptides, antibodies, aptamers, nucleic acids, and proteins. For example, activation of BCKDH may be achieved by RNA interference (e.g. siRNA, shRNA, miRNA, or saRNA). For example, activation of BCKDH may be achieved by RNA interference against transcripts encoding proteins that regulate BCKDH activity. In some embodiments, BCKDH agonists may be antibodies known to activate BCKDH. For example, BCKHD agonists may be antibodies known to inhibit BDK kinase and or activate PPM1K.

In some embodiments, BCKHD agonists may be small molecules known to activate BCKDH. In particular embodiments, suitable BCKDH agonists may be BDK kinase inhibitors. Suitable BDK kinase inhibitors include, for example, benzothiophene carboxylate derivates. Suitable benzothiophene carboxylate derivatives include cholorophenylproprionate (CPP) (for example, (S)-α-cholorophenylproprionate ((S)-CPP)), (N-(4-amino-1,2,5-oxadiazol-3-yl)-3,6-dichlorobenzo[b]thiophene-2-carboxamide) (BT1), (3,6-dichlorobenzo[b]thiophene-2-carboxylic acid) (BT2), (3-chloro-6-fluorobenzo[b]thiophene-2-carboxylic acid) (BT2F), and (N-(4-acetamido-1,2,5-oxadiazol-3-yl)-3,6-dichlorobenzo[b]thiophene-2-carboxamide) (BT3). For example, the BCKDH agonist may be the small molecule BDK kinase inhibitor BT2.

In some embodiments, suitable BCKDH agonists may be PPM1K agonists. In some embodiments, one or more BDK kinase inhibitors and one or more PPM1K agonists may be used.

In some embodiments, the one or more BCKDH agonists may be combined with other known therapies for the treatment of NAFLD, including antioxidants, cytoprotective agents, antidiabetic agents, insulin-sensitizing agents, anti-hyperlipidemic agents, acetyl co-A carboxylase inhibitors, ATP-citrate lyase inhibitors, and surgery. For example, the one or more BCKDH agonists may be combined with bariatric surgery.

In accordance with any of the embodiments described herein, therapeutic agents may be administered by themselves or as a part of a pharmaceutical composition comprising the one or more therapeutic agents and one or more carriers. Suitable carriers depend on the intended route of administration to the subject. Contemplated routes of administration include those oral, rectal, nasal, topical (including transdermal, buccal and sublingual), vaginal, parenteral (including subcutaneous, intramuscular, intravenous and intradermal) and pulmonary administration. In some embodiments, the composition or compositions are conveniently presented in unit dosage form and are prepared by any method known in the art of pharmacy. Such methods include the step of bringing into association the active ingredient with the carrier which constitutes one or more accessory ingredients. In general, the formulations are prepared by uniformly and intimately bringing into association (e.g., mixing) the active ingredient with liquid carriers or finely divided solid carriers or both, and then if necessary shaping the product.

Formulations of the present disclosure suitable for oral administration may be presented as discrete units such as capsules, cachets or tablets, wherein each preferably contains a predetermined amount of the one or more therapeutic agents as a powder or granules; as a solution or suspension in an aqueous or non-aqueous liquid; or as an oil-in-water liquid emulsion or a water-in-oil liquid emulsion. In other embodiments, the composition is presented as a bolus, electuary, or paste, etc.

Preferred unit dosage formulations are those containing a daily dose or unit, daily subdose, or an appropriate fraction thereof, of an agent.

It should be understood that in addition to the ingredients particularly mentioned above, the compositions may include other agents conventional in the art having regard to the route of administration in question. For example, compositions suitable for oral administration may include such further agents as sweeteners, thickeners and flavoring agents. Still other formulations optionally include food additives (suitable sweeteners, flavorings, colorings, etc.), phytonutrients (e.g., flax seed oil), minerals (e.g., Ca, Fe, K, etc.), vitamins, and other acceptable compositions (e.g., conjugated linoelic acid), extenders, preservatives, and stabilizers, etc.

Various delivery systems are known and can be used to administer compositions described herein, e.g., encapsulation in liposomes, microparticles, microcapsules, receptor-mediated endocytosis, and the like. Methods of delivery include, but are not limited to, intra-arterial, intra-muscular, intravenous, intranasal, and oral routes. In specific embodiments, it may be desirable to administer the compositions of the disclosure locally to the area in need of treatment; this may be achieved by, for example, and not by way of limitation, local infusion during surgery, injection, or by means of a catheter.

Therapeutic amounts are empirically determined and vary with the pathology being treated, the subject being treated and the efficacy and toxicity of the agent. It is understood that therapeutically effective amounts vary based upon factors including the age, gender, and weight of the subject, among others. It also is intended that the compositions and methods of this disclosure be co-administered with other suitable compositions and therapies.

In general, suitable doses of the therapeutic agent may range from about 1 ng/kg to about 1 g/kg. For example, a suitable dose may be from about 1 ng/kg to about 1 g/kg, about 100 ng/kg to about 900 mg/kg, about 200 ng/kg to about 800 mg/kg, about 300 ng/kg to about 700 mg/kg, about 400 ng/kg to about 600 mg/kg, about 500 ng/kg to about 500 mg/kg, about 600 ng/kg to about 400 mg/kg, about 700 ng/kg to about 300 mg/kg, about 800 ng/kg to about 200 mg/kg, about 900 ng/kg to about 100 mg/kg, about 1 μg/kg to about 50 mg/kg, about 10 μg/kg to about 10 mg/kg, about 100 μg/kg to about 1 mg/kg, about 200 μg/kg to about 900 μg/kg, about 300 μg/kg to about 800 μg/kg, about 400 μg/kg to about 700 μg/kg, or about 500 μg/kg to about 600 μg/kg.

The one or more therapeutic agents may be administered to the subject at any desired frequency. For example, the one or therapeutic agents may be administered to the subject more than once per day (e.g. twice per day, three times per day, four times per day, and the like), once per day, once every other day, once a week, and the like. The one or more therapeutic agents may be provided to the subject for any desired duration. For example, the one or more therapeutic agents may be administered to the subject for at least one week, at least two weeks, at least three weeks, at least one month, at least two months, at least three months, at least six months, at least one year, at least two years, at least three years, at least four years, at least five years, at least ten years, at least twenty years, or for the lifetime of the subject.

The present disclosure also provides kits comprising a therapeutic agent as disclosed herein.

The following examples further illustrate the invention but, of course, should not be construed as in any way limiting its scope.

### Example 1

The present example demonstrates that DNL is regulated in part by the levels of the branched chain α-keto acid dehydrogenase kinase (BDK) and phosphatase (PPM1K), previously known only for their role regulating the rate of branched chain α-keto acid (BCKA) catabolism by the BCKA dehydrogenase (BCKDH) complex. BDK and PPM1K exert their control over hepatic DNL by directly modulating the phosphorylation state of ATP-citrate lyase (ACL). Whereas phosphorylation of BCKDH is inhibitory and leads to accumulation of BCKA in plasma, phosphorylation of ACL is activating and results in increased DNL by virtue of the production of cytosolic acetyl CoA and then malonyl CoA from citrate. In both animal models and humans, hepatic BDK levels are elevated in obesity and by ingestion of diets high in fructose, whereas PPM1K levels are low in these settings and increased during fasting. Importantly, adenovirus-mediated overexpression of recombinant BDK in liver is sufficient to raise DNL by 2.5-fold in lean healthy rats. In contrast, inhibition of BDK with a small molecule, BT2, or adenovirus-mediated overexpression of recombinant PPM1K in liver of obese Zucker fatty rats potently lowers circulating BCKA levels and hepatic triglyceride content by >40% within seven days in the absence of changes in food intake or weight gain.

This example evaluated the potential therapeutic impact of manipulation of the BCKDH complex and its regulatory kinase, BDK, and phosphatase, PPM1K. Taken together, the results presented herein demonstrate that manipulation of the BCKDH complex represents a viable therapeutic option for the treatment of NAFLD.

### Experimental Model and Subject Details

Animal Studies: All animal procedures were approved by and carried out in accordance with the policies of the Duke University Institutional Animal Care and Use Committee. Rats were housed in a 12-hour light: dark cycle and given ad-libitum access to food and water for the duration of the study unless stated otherwise. All rats were euthanized by cardiac puncture after being anesthetized with Nembutal (80 mg/kg) administered by intraperitoneal (i.p.) injection. Tissues and plasma were rapidly harvested and snap frozen in liquid nitrogen for biochemical analyses.

Individually housed, male 12 week-old Zucker fatty rats (ZFR, Charles River Laboratories) maintained on a custom control low fat (LF) diet (A11072001, Research Diets) were used for the BDK inhibition and PPM1K overexpression studies. For the BDK inhibition study, ZFR were administered the small molecule BDK inhibitor 3,6-dichlorobenzo[b]thiophene-2-carboxylic acid (BT2, Sigma) daily at a dose of 20 mg/kg dissolved in 200 ul of sterile dimethylsulfoxide by i.p. injection for 7 days. The control group was administered an equal volume of vehicle each day. On day 6, following an overnight fast, ZFR were subjected to a 1 g/kg i.p. glucose tolerance test (GTT) precisely 1 hr after administration of BT2 (20 mg/kg i.p.) or vehicle. The glucose tolerance test was performed as described (White, et al., 2016). Following the GTT, rats were returned to their normal cages and provided free access to food and water. Indirect calorimetry was performed in a second cohort of BT2 treated rats on day 6 of BT2 administration. Here ad-libitum fed rats were injected with BT2 (20 mg/kg i.p.) or vehicle immediately prior to being placed in an eight-chamber Oxymax system (Columbus Instruments) for seven hours. During their time in the metabolic cages all rats had free access to food and water. The next morning, rats were euthanized in the fed state precisely 1 hr following a final dose of BT2 (20 mg/kg i.p.) or vehicle.

For the PPM1K overexpression study, male 12 week-old ZFR were administered two doses of cyclosporine (15 mg/kg i.p., Novartis) prior to adenovirus administration. The first dose of cyclosporine was given 24 hours prior and the second dose was administered immediately prior to tail vein injection of Ad-CMV-PPM1K or Ad-CMV-GFP adenovirus (2×10¹² viral particles per kg). Both viruses use the CMV promoter to drive transgene expression. As for the BT2 study described above, ZFR were subjected to a 1 g/kg i.p. GTT on the 6^{th} day following administration of adenovirus and euthanized the next morning in the ad-libitum fed state.

Dual housed, 8-week old male Wistar rats (Charles River Laboratories) maintained on a standard chow diet (TD.7001, Harlan Teklad) were used for the BDK and ChREBP overexpression, fructose refeeding, and subcellular fractionation studies. To achieve BDK overexpression, rats were transfected with recombinant adenoviruses encoding a V5 tagged BDK (Ad-CMV-BDK) or β gal (Ad-CMV-□GAL), driven by the CMV promoter as described for the PPM1K study above. Five days after virus administration rats were injected with a bolus of sterile ²H₂O (10 μl/g, Sigma) containing 0.09% NaCl (w/v) and maintained on drinking water containing 4% ²H₂O for the remainder of the study. Rats were euthanized 2 days later in the ad-lib fed state and liver was snap frozen in liquid nitrogen. For ChREBP overexpression studies, rats were treated with recombinant adenoviruses containing the mouse (m) ChREBP-β (Ad-CMV-ChREBP-β or GFP (Ad-CMV-GFP) cDNAs, driven by the CMV promoter. Seven days after virus administration, rats were sacrificed as described for the BDK study. To study the effect of fructose refeeding a separate cohort of untreated rats were fasted overnight and then refed with either standard chow or a high fructose diet (TD.89247, Harlan Teklad) containing 60% fructose. Four hours later rats were euthanized. For fractionation studies, rats were euthanized following a 20 hour fast or in the ad-libitum fed state and a 1 cm³ portion of the right lobe of the liver was placed in KMEM buffer on ice for subsequent fractionation. The rest of the lobe was snap frozen in liquid nitrogen for subsequent analysis of ACL phosphorylation.

Human samples: cDNA was obtained to measure BDK and PPM1K expression in human liver samples. These human liver samples were derived from a subgroup of patients enrolled in an NAFLD registry at Beth Israel Deaconess Medical Center (BIDMC) beginning in 2009, which is a prospective study that enrolls subjects with biopsy-proven NAFLD. Use of human liver samples was approved by the BIDMC institutional research board.

Cell culture studies: Fao hepatoma cells (Sigma) were cultured in RPMI-1640 (Gibco) containing 10% FBS (Sigma). 24 hours after plating, cells were incubated for 18 hours with individual adenoviruses at approximately 8×10⁸ viral particles per mL, and samples were harvested 72 hours later for immunoblot analyses. Human embryonic kidney (HEK) 293 cells were used to visualize localization of CMV-ΔMTS-BDK-GFP and control CMV-GFP constructs by confocal microscopy. Cells were plated and transfected in a 96 well glass bottom plate that had been pre-coated with poly-D lysine solution for 1-hour at room temperature. For transfection, cells were incubated in Opti-mem containing 1.5 μl of Mirus, TransIT-293 transfection reagent and 1 μg DNA per well. After 24 hours, mitochondrial and nuclear staining was performed in live cells using Image-IT Live Mito and nuclear labeling Kit (Cell permeant MitoTracker Red CMXRos (579/599 nm) and Hoechst 33342 (350/461 nm) ThermoFisher). Confocal images were captured using a Zeiss LSM 510 inverted confocal microscope using a 405 diode for Hoechst, Argon for GFP, and HeNe 561 for MitoTracker. Images were captured in one plane using a 63× oil objective. Each wavelength was acquired separately and then consolidated after acquisition.

Method Details

Adenoviral reagents: Recombinant Ad-CMV-PPM1K and Ad-CMV-GFP adenoviral stocks were purchased from Vector Biolabs. pAd/CMV/V5-DEST containing β gal cDNA was purchased from Thermo Life. Gateway pDONR223 plasmids containing cDNAs encoding BDK (BCKDK, HsCD00511364; includes the N-terminal mitochondrial targeting pre-sequence) and ACL (ACLY, HsCD00399238) were purchased from DNASU (Seiler et al., 2014) and recombined into pAd/CMV/V5-DEST per the manufacturer's protocol. Recombined adenovirus plasmids were linearized with PacT (NEB) and transfected into HEK293 cells to generate adenoviral stocks. Murine 3×-Flag-ChREBP-beta was cloned into the pShuttle-IRES-hrGFP-1 vector (Agilent) and adenovirus was generated with both the empty and ChREBP vectors using the AdEasy Adenoviral Vector System (Agilent). Adenoviruses and expression vectors for Δ-MTS-BDK were generated using a new modular cloning platform, pMVP. Briefly, cDNA for BDK devoid of the MTS was amplified from HsCD00511364 (DNASU) without a stop codon by PCR and subsequently recombined into pDONR221 P4r-P3r (Invitrogen) using BP Clonase II per the manufacturer's protocol (Invitrogen) to form a Gateway entry plasmid, pENTR R4-R3/cBDK. Amplification was performed using the following primers:

Forward primer:
(SEQ ID NO: 1)
GGGGACAACTTTTCTATACAAAGTTGCCATGGCTTCGACGTCGGCC
ACCGA
Rev. primer:
(SEQ ID NO: 2)
GGGGACAACTTTATTATACAAAGTTGTGATCCGGAAGCTTTCCTCC

The expression vector Δ-MTS-BDK-GFP was made by recombination of pENTR R4-R3/cBDK with custom Multisite Gateway Pro entry plasmids containing elements encoding the (1) the CMV promoter and (2) GFP followed by the SV40 polyadenylation signal, into a custom Gateway destination plasmid, pMVPBS-DEST, mediated by LR Clonase II plus (Invitrogen). The GFP control plasmid was generated by LR Clonase II plus-mediated recombination of GFP into pEF-DEST51 (Invitrogen) per the manufacturer's instructions. The adenoviral vectors Ad-CMV-GFP and Ad-CMV-Δ-MTS-BDK were created by recombination of pENTR R4-R3/cBDK or pENTR R4-R3/GFP with custom Multisite Gateway Pro plasmids encoding (1) the CMV promoter and (2) a 3×-HA epitope tag followed by the bGH polyadenylation signal, into the pAd/PL-DEST adenovirus vector (Invitrogen). Recombinant adenoviral plasmids were linearized with PacI, and propagated in HEK293 cells. All recombinant adenoviruses were amplified in HEK293 cells and purified using CsCl₂ gradients, titered by A260, and determined to be E1A deficient using a qRT-PCR screen (Jensen et al., 2013; Lavine et al., 2010).

BCKDH activity assay: Tissue BCKDH activity was measured. Briefly, frozen tissue samples were pulverized in liquid nitrogen, then homogenized using a QIAGEN TissueLyser II in 250 μl of ice cold buffer I (30 mM KPi pH 7.5, 3 mM EDTA, 5 mM DTT, 1 mM α-ketoisovalerate, 3% FBS, 5% Triton X-100, 1 μM Leupeptin). Samples were then centrifuged for 10 min at 10,000×g and 50 μL of supernatant was added to 300 μL of buffer II (50 mM HEPES pH 7.5, 30 mM KPi pH 7.5, 0.4 mM CoA, 3 mM NAD+, 5% FBS, 2 mM Thiamine Pyrophosphate, 2 mM MgCl2, 7.8 μM α-keto [1-¹⁴C] isovalerate) in a polystyrene test tube containing a raised 1 M NaOH CO₂ trap. Tubes were capped and placed in a shaking water bath at 37° C. for 30 min. The reaction mixture was acidified by injection of 70% perchloric acid followed by shaking on an orbital shaker for 1 h. The ¹⁴CO₂ contained in the trap was counted in a Beckman Coulter LS6500 liquid scintillation counter.

Metabolite profiling: Amino acids were measured in plasma and liver samples, and acylcarnitines in liver samples. Methods of sample handling and extraction have been described previously (Ferrara et al., 2008; Ronnebaum et al., 2006). Amino acid and acylcarnitine profiling was performed by tandem mass spectrometry (MS/MS) (Ferrara et al., 2008; Newgard et al., 2009). All MS analyses employed stable-isotope-dilution with internal standards from Isotec, Cambridge Isotopes Laboratories, and CDN Isotopes. A list of all internal standards used in these studies has been published previously (Ferrara et al., 2008; Newgard et al., 2009).

Plasma concentrations of the alpha-keto acids of leucine (α-keto-isocaproate, KIC), isoleucine (α-keto-β-methylvalerate, KMV) and valine (α-keto-isovalerate, KIV) were measured by LC-MS as previously described (Glynn et al., 2015; White et al., 2016). Other plasma analytes were measured on a Beckman DxC600 autoanalyzer, using reagents for lactate, total cholesterol, and triglycerides from Beckman, and non-esterified fatty acids (NEFA) and ketones (total and 3-hydroxybutyrate) from Wako (Richmond, Va.). Glycerol was measured using reagents from TG-B by Roche Diagnostics (Indianapolis, Ind.). Liver triglycerides were quantified using the triglyceride quantification kit from Abcam. Plasma insulin concentrations were measured with a Millipore EMD Rat insulin ELISA kit.

Phosphoproteomics: Large-scale measurements of phosphorylation changes in response to BT2 and PPM1K were performed. Briefly, protein from livers of ZFR treated with BT2 or DMSO, or with Ad-CMV-PPM1K or Ad-CMV-GFP, were solubilized, digested with LysC and trypsin, labeled with TMT-6plex reagents, and mixed in batches of six to enable two direct comparisons: 1) BT2 vs. DMSO (n=3), 2) PPM1K vs. GFP (n=3). Phosphopeptides were enriched from the majority of each mixture using immobilized metal affinity chromatography (IMAC), and a small portion of the input material was retained for assessment of relative protein abundance. Both phosphopeptide and input fractions were subjected to nanoLC-MS/MS using a nano-Acquity UPLC system (Waters) coupled to a Q Exactive Plus Hybrid Quadrupole-Orbitrap mass spectrometer (Thermo Fischer Scientific). Raw LC-MS/MS data were processed in Proteome Discoverer v2.1 (PD2.1, Thermo Fisher Scientific) and subsequent statistical analysis was performed in Microsoft EXCEL. The precise localization of the phosphosites found to be significantly altered by each intervention was validated in Proteome Discoverer v2.2. (PD2.2, Thermo Fisher Scientific). Phosphosite motif analysis and logo generation was performed in PhosphoSitePlus® by submitting pre-aligned 15 amino acid sequences for all modulated phosphopeptides from each study into the motif and logo analysis tools (Hornbeck et al., 2015).

Tissue lysis and protein digestion for proteomics: Approximately 50 mg of pulverized liver tissue power from each rat (3 BT2- and 3 DMSO-treated animals; 3 PPM1K- and 3 GFP-overexpressing animals) was re-suspended in 400 μL of ice-cold 8M Urea Lysis Buffer (8 M urea in 50 mM Tris, pH 8.0, 40 mM NaCl, 2 mM MgCl₂, 1 mM Na₃VO₄, 10 mM Na₄P₂O₇, 50 mM NaF, supplemented with protease inhibitor (1× cOmplete mini EDTA-free) and phosphatase inhibitor (1× PhosStop) tablets). Samples were lysed with a TissueLyzer for 30 seconds at 30 Hertz twice and the tissue was further disrupted by sonication with a probe sonicator in three 5 second bursts (power setting of 3), incubating on ice in-between each burst. Samples were centrifuged at 10,000×g for 10 min at 4° C. and the supernatant was retained. Protein concentration was determined by BCA, and equal amounts of protein (500 μg, adjusted to 2.5 mg/mL with Urea Lysis Buffer) from each sample was reduced with 5 mM DTT at 37° C. for 30 min, cooled to RT, alkylated with 15 mM iodoacetamide for 30 min in the dark and unreacted iodoacetamide quenched by the addition of DTT up to 15 mM. Each sample was digested with 5 μg LysC (100:1 w/w, protein to enzyme) at 37° C. for 4 hours. Following dilution to 1.5 M urea with 50 mM Tris (pH 8.0), 5 mM CaCl₂), the samples were digested with trypsin (50:1 w/w, protein:enzyme) overnight at 37° C. The samples were acidified to 0.5% TFA and centrifuged at 4000×g for 10 min at 4° C. to pellet insoluble material. The supernatant containing soluble peptides was desalted by solid phase extraction (SPE) with a Waters 50 mg tC18 SEP-PAK SPE column and eluted once with 500 μL 25% acetonitrile/0.1% TFA and twice with 500 μl 50% acetonitrile/0.1% TFA. The 1.5 ml eluate was frozen and dried in a speed vac.

Peptide labeling and PTM enrichment: Each peptide sample was re-suspended in 100 μL of 200 mM triethylammonium bicarbonate (TEAB), mixed with a unique 6-plex Tandem Mass Tag (TMT) reagent (0.8 mg re-suspended in 50 μL 100% acetonitrile), using one TMT kit (reagents 126-131) for the BT2vDMSO comparison (n=3) and a separate TMT kit for the PPM1KvGFP comparison (n=3). Samples were shaken for 4 hours at room temperature and subsequently quenched with 0.8 μL 50% hydroxylamine and shaken for 15 additional minutes at room temperature. For both experiments (BT2vDMSO, PPM1KvGFP), 2.5 uL of each of the six samples was mixed for QC analysis. After subjecting the QC samples to the LC-MS/MS workflow described below, the data was searched as described below, but with TMT labeling as a variable modification on the peptide N-terminus to assess TMT labeling efficiency—which was determined to be 94.6% (BT2vDMSO) and 91.3% (PPM1KvGFP)—and total peptide ratios. For each study, the remainder of all six samples were combined with slight adjustments for any deviation from 1:1:1:1:1:1 ratios, frozen, and dried in a speed vac. The TMT-labeled peptide mixtures for each experiment (BT2vDMSO, PPM1KvGFP) were re-suspended in 1 mL 0.5% TFA and subjected to SPE again with a Waters 100 mg tC18 SEP-PAK SPE column as described above. For both experiments, the eluate was vortexed and split into one aliquot containing ˜5% of the total peptide mixture (150 μg) and a second aliquot containing ˜95% (2.85 mg). Both aliquots were frozen and dried in a speed vac. The 150 μg aliquot of the “input” material was saved at −80° C. for quantification of unmodified peptides. The 2.85 mg aliquot was subjected to phosphopeptide enrichment via immobilized metal affinity chromatography (IMAC) using Ni-NTA Magnetic Agarose Beads, as described previously (51) with slight modifications. Briefly, the beads were washed three times with water, incubated in 40 mM EDTA, pH 8.0 for 30 minutes while shaking, and subsequently washed with water three times. The beads were then incubated with 100 mM FeCl₃ for 30 minutes while shaking, and were washed four times with 80% acetonitrile/0.15% TFA. Samples were re-suspended in 1 ml 80% acetonitrile/0.15% TFA, added to the beads, and incubated for 30 minutes at room temperature while shaking. Samples were subsequently washed three times with 1 ml 80% acetonitrile/0.15% TFA and eluted for 1 minute by vortexing in 100 μl of 50% acetonitrile, 0.7% NH₄OH. Eluted phosphopeptides were acidified immediately with 50 μl 4% formic acid, frozen and dried in a speed vac.

Nano-LC-MS/MS for TMT proteomic experiment: All samples were submitted to the Duke University School of Medicine Proteomics Core facility for analysis by nanoLC-MS/MS analysis using a nano-Acquity UPLC system (Waters) coupled to a Q Exactive Plus Hybrid Quadrupole-Orbitrap mass spectrometer (Thermo Fischer Scientific) via a nanoelectrospray ionization source. Prior to injection, the phosphopeptide samples were re-suspended in 12 μL 0.1% formic acid supplemented with 10 mM citrate. Each phosphopeptide sample was analyzed by 1D LC-MS/MS with technical replicate analysis with 1 μL of sample injected (˜2 hr runs) and by a single 2D LC-MS/MS (5 high pH reversed phase fractions, subjected to ˜2 hr runs) with the remainder of the sample injected. For each experiment (BT2vDMSO, PPM1KvGFP), the input material described above (5% of the large-scale mixture, ˜150 μg of TMT-labeled peptides) was subjected to high pH reversed phase pre-fractionation for 2D LC-MS/MS twice, once using 5 fractions (subjected to ˜45 min LC-MS/MS runs each) and once using 9 fractions (subjected to ˜3 hr runs each). For each injection, the sample was first trapped on a Symmetry C18 20 mm×180 μm trapping column (5 μl/min at 99.9/0.1 v/v water/acetonitrile), after which the analytical separation was performed over a 90 minute gradient (flow rate of 400 nanoliters/minute) of 3 to 30% acetonitrile using a 1.7 μm Acquity BEH130 C18 75 μm×250 mm column (Waters Corp.), with a column temperature of 55° C. MS' (precursor ions) was performed at 70,000 resolution, with an AGC target of 1×10⁶ ions and a maximum injection time of 60 ms. MS² spectra (product ions) were collected by data-dependent acquisition (DDA) of the top 20 most abundant precursor ions with a charge greater than 1 per MS1 scan, with dynamic exclusion enabled for a window of 30 seconds. Precursor ions were filtered with a 1.2 m/z isolation window and fragmented with a normalized collision energy of 30. MS2 scans were performed at 17,500 resolution, with an AGC target of 1×10⁵ ions and a maximum injection time of 60 ms.

Data processing for TMT proteomic experiment: Raw LC-MS/MS data were processed in Proteome discoverer v2.1 (PD2.1, Thermo Fisher Scientific), using both the Sequest HT and MS Amanda search engines. Data were searched against the UniProt rat complete proteome database of reviewed (Swiss-Prot) and unreviewed (TrEMBL) proteins, which consisted of 29,885 sequences on the date of download (Dec. 29, 2015). Default search parameters included oxidation (15.995 Da on M) as a variable modification and carbamidomethyl (57.021 Da on C) and TMTplex (229.163 Da on peptide N-term and K). Phospho runs added phosphorylation (79.966 Da on S,T,Y) as a variable modification. Data were searched with a 10 ppm precursor mass and 0.02 Da product ion tolerance. The maximum number of missed cleavages was set to 2 and enzyme specificity was trypsin (full). Considering each data type (phospho, input) separately, peptide spectral matches (PSMs) from each search algorithm were filtered to a 1% false discovery rate (FDR) using the Percolator node of PD2.1. For phospho data, site localization probabilities were determined for all modifications using the ptmRS algorithm. PSMs were grouped to unique peptides while maintaining a 1% FDR at the peptide level and using a 95% site localization threshold for phosphorylation. Peptides from all samples (phosho, input) were grouped to proteins together using the rules of strict parsimony and proteins were filtered to 1% FDR using the Protein FDR Validator node of PD2.1. Reporter ion intensities for all PSMs having co-isolation interference below 0.25 (25% of the ion current in the isolation window) and average reporter S/N>10 for all reporter ions were summed together at the peptide group and protein level, but keeping quantification for each data type (phosho, input) and experiment (BT2vvehicle, PPM1KvGFP) separate. Peptides shared between protein groups were excluded from protein quantitation calculations.

Statistical analysis for TMT proteomic experiment: Protein and peptide groups tabs in the PD2.1 results were exported as tab delimited .txt. files, opened in Microsoft EXCEL, and analyzed. First, peptide group reporter intensities for each peptide group in the input material were summed together for each TMT channel, each channel's sum was divided by the average of all channels' sums, resulting in channel-specific loading control normalization factors to correct for any deviation from equal protein/peptide input into the six sample comparison. Reporter intensities for peptide groups from the phospho fractions and for proteins from the input fraction were divided by the loading control normalization factors for each respective TMT channel. Analyzing the phosphopeptide and protein datasets separately, all loading control-normalized TMT reporter intensities were converted to log₂ space, and the average value from the six samples was subtracted from each sample-specific measurement to normalize the relative measurements to the mean. For the BT2 vs. vehicle and the PPM1K vs. GFP comparisons (n=3), condition average, standard deviation, p-value (p, two-tailed student's t-test, assuming equal variance), and adjusted p-value (P_{adjusted}, Benjamini Hochberg FDR correction) were calculated. For protein-level quantification, only Master Proteins—or the most statistically significant protein representing a group of parsimonious proteins containing common peptides identified at 1% FDR—were used for quantitative comparison.

Fractionation: Mitochondrial and cytosolic fractions were isolated from liver samples by differential centrifugation. Tissues were homogenized in KMEM buffer (100 mM KCl, 50 mM MOPS, 1 mM EGTA, 5 mM MgSO4, 0.2% BSA) with a Teflon pestle and centrifuged at 500×g to remove cell debris. The supernatants were then centrifuged at 9000×g to pellet mitochondria. The supernatant containing cytosolic proteins was then transferred to a new tube and subjected to an additional three rounds of centrifugation at 9000×g to ensure full removal of mitochondria from the fraction. The mitochondrial pellet was also resuspended and subjected to centrifugation at 9000×g an additional two times. The pellet was then resuspended in 500 μl of KMEM and protein concentration of both fractions was assayed using a BCA kit.

Immunoblotting: Tissue lysates used for immunoblotting were prepared in Cell Lysis Buffer (Cell Signaling Technologies) containing protease inhibitor tablets (Roche), phosphatase inhibitor cocktails 2 and 3 (Sigma), and 10 mM PMSF. 50 μg of protein was loaded onto a 4-12% Bis-Tris gel (Novex), subjected to SDS-PAGE, and then transferred onto PVDF membranes. Membranes were blocked and then probed with the appropriate antibodies. All primary antibodies were used at a concentration of 1:1000. Secondary antibodies were diluted 1:10000. All antibodies used are listed in the key resources table. Immunoblots were developed using a Li-Cor Odyssey CLx and quantified using the Li-Cor software.

Assays for incorporation of [γ-³²P]-phosphoryl group into ACL: The phosphorylation reaction mixture in a total volume of 16 μl contained 20 mM Tris-Cl (pH 7.5), 100 mM KCl, 5 mM MgCl₂, 2 mM DTT, 0.02% (v/v) Tween-20, and 0.1 mg/ml bovine serum albumin. To the reaction mixture, the following various combinations of recombinant proteins were added: 1 μg 147 kDa GST-tagged human ACL (Sigma), 2.6 μg BCKD E1 (an □2□2 heterotetramer), 1.5 μg BCKD E2, 0.3 μg 40 kDa catalytic subunit of bovine PKA (Promega) and 0.3 μg MBP-BDK (mature sequence only: amino acid residue 31-382) (Davie et al., 1995). Reaction mixtures without either kinase served as controls. [γ³²P] ATP (200-300 cpm/pmol) was added to a final 300 μM concentration to initiate the kinase reaction. After incubation at room temperature for 10 min, the reaction was stopped by adding 4 μl SDS-PAGE sample buffer, followed by a second incubation at 100° C. for κ min. The reaction products were analyzed by SDS gel electrophoresis in 12% acrylamide. Radioactivity on the gel was analyzed by exposing the gel on a storage phosphor plate overnight and scanning the autoradiograph in a Typhoon imager.

To confirm that BDK phosphorylates ACL on serine 454, a complementary phosphorylation assay was performed using combinations of freshly purified VS-tagged BDK, VS-tagged β gal, and VS-tagged ACL that were isolated from Fao hepatoma cell lysates that had been transfected with the respective adenoviruses as described above. V5 tagged proteins were purified from pooled Fao cell lysates using the V5-immunoprecipitation kit from MBP. Phosphorylation reactions were performed in 50 μl of reaction mixture containing 20 mM Tris-Cl (pH 8.0), 10 mM MgCl₂, 10 mM glycerophosphate, 0.1 mg/ml bovine serum albumin, and 10 mM ATP. The reaction was performed at room temperature for 10 minutes and was stopped by the addition of SDS-PAGE sample buffer, followed by incubation at 100° C. for 5 minutes. Phosphorylation of ACL on serine 454 was detected by immunoblot.

²H₂O label quantitation: Plasma ²H₂O enrichment and total palmitic acid labeling in the liver was assayed. Briefly, for plasma ²H₂O labeling, 10 μl plasma or standard was mixed with 2 μl of a 10 M NaOH solution and 4 μl of acetone/acetonitrile solution (1/20, volume ratio). Samples were mixed gently and incubated overnight. The acetone was then extracted by adding 500 μl chloroform. The chloroform phase was dried by addition of ˜50 mg NaSO₄ salt, and then 100 μl of the chloroform layer underwent GC-MS analysis using an Agilent 5973N-MSD equipped with an Agilent 6890 GC system, and a DB-17MS capillary column (30 m×0.25 mm×0.25 μm). The mass spectrometer was operated in the electron impact mode (EI; 70 eV). The temperature program was as follows: 60° C. initial, increase by 20° C./min to 100° C., increase by 50° C./min to 220° C., and hold for 1 min. The sample was injected at a split ratio of 40:1 with a helium flow of 1 ml/min. Acetone eluted at 1.5 min. Selective ion monitoring of mass-to-charge ratios of 58 and 59 was performed using a dwell time of 10 ms/ion.

For total palmitic acid labeling in liver, 20 mg liver tissue was homogenized in 1 ml KOH/EtOH (EtOH 75%) and incubated at 85° C. for 3 hours. 200 μl of internal standard [¹³C-16]palmitate was added into samples after cooling. 100 μl of sample was acidified by addition of equal volume of 6 M HCl. Palmitic acid was extracted in 600 μl chloroform. The chloroform layer was completely dried by nitrogen gas and reacted with 50 μl N-methyl-N-trimethylsilylfluoroacetamide (TMS) at 70° C. for 30 minutes. TMS derivative was analyzed by GC-MS using an Agilent 5973N-MSD equipped with an Agilent 6890 GC system, and a DB-17MS capillary column (30 m×0.25 mm×0.25 μm). The mass spectrometer was operated in the electron impact mode (EI; 70 eV). The temperature program was as follows: 100° C. initial, increase by 15° C./min to 295° C. and hold for 8 min. The sample was injected at a split ratio of 10:1 with a helium flow of 1 ml/min. Palmitate-TMS derivative eluted at 9.7 min. Mass scan from 100 to 600 was chosen in the method. The m/z at 313, 314, and 319 were extracted for M0, M1, and M16 palmitate quantitation.

Stable isotope labeling was corrected from the natural stable isotope distribution (Tomcik et al., 2011). Newly synthesized total palmitic acid was calculated as % newly synthesized palmitic acid labeling=total palmitic acid labeling/(plasma ²H₂O labeling×22)×100.

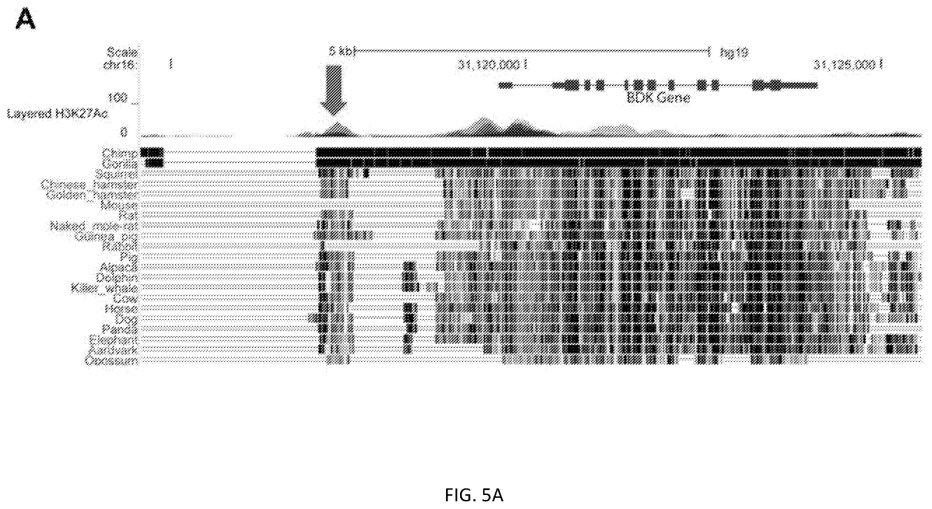

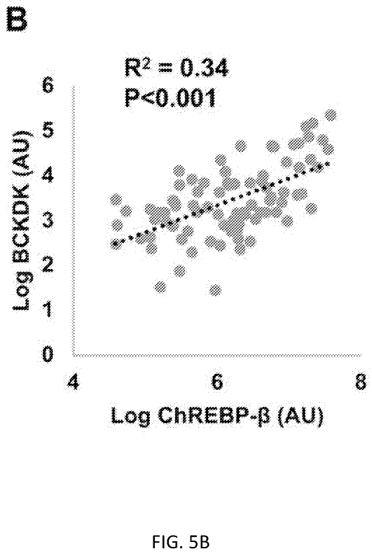

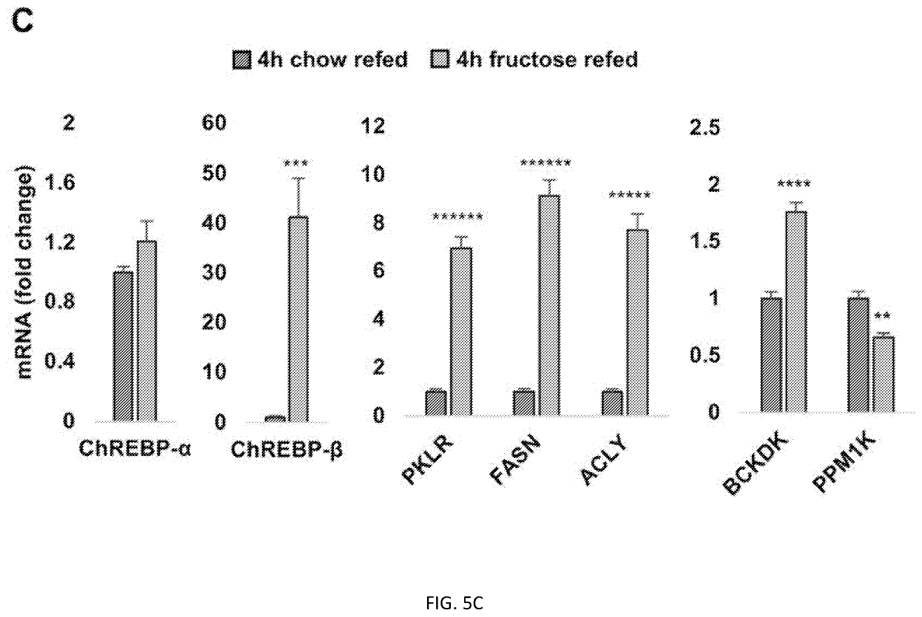

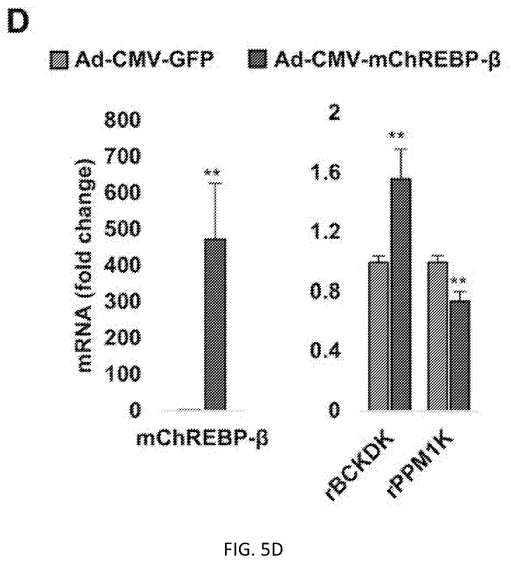

Transcriptomic analyses by qPCR: For detection of human and rat PPM1K mRNA expression in the PPM1K study, RNA was extracted from liver tissue using an RNeasy kit from QIAGEN. RNA was reverse transcribed using the Bio-Rad iScript cDNA synthesis kit. qPCR was performed with Applied Biosystems TaqMan® gene expression assays for hPPM1K (Hs00410954_m1), rPPM1K (Rn01410038_m1), and rPPIA (Rn00690933_m1) on a Viia 7 Real-Time PCR system (Applied Biosystems). Each sample was run in duplicate and normalized to Ppia. For human, high fructose, and ChREBP overexpression studies, TM reagent (MRC, catalog TR118) was used for RNA isolation. RNA was reverse transcribed using a SuperScript VILO kit (Invitrogen). Gene expression was analyzed with the ABI Prism sequence detection system (SYBR Green; Applied Biosystems). Gene-specific primers were synthesized by IDT. Each sample was run in duplicate, and normalized to Rp1p0 RNA. Primers used are listed in the key resources table. Human liver samples used for qPCR analysis as shown in FIG. 5B were from a subgroup of 86 patients (49 male, 37 female, age range 18-83 years; median age 52 years) with biopsy-proven NAFLD enrolled in a NAFLD registry at Beth Israel Deaconess Medical Center (BIDMC). The study was approved by the BIDMC institutional review board and was conducted in accordance with the Helsinki declaration of 1975, as revised in 1993. All participants consented to the study upon enrollment.

Quantification and statistical analysis: All data are expressed as mean±SEM. Results from animal and cell studies were analyzed using a two-way Student's t-test. Regression analysis of human qPCR data was performed with SPSS release 18.0.0. A p value less than 0.05 was considered statistically significant.

Data and software availability: Raw LC-MS/MS proteomics data have been deposited to the ProteomeXchange Consortium via the PRIDE partner repository, see key resources table for project accession number.

TABLE 1
Key resources table

| | | |
|---|---|---|
| REAGENT or RESOURCE | SOURCE | IDENTIFIER |

Antibodies

| | | |
|---|---|---|
| pACL ser454/455 | Cell signaling | CS43315 |
| | technologies | |
| ACL | Thermo Fisher | PA5-29495 |
| pAKT ser473 | Cell signaling | 9271 |
| | technologies | |
| AKT | Cell signaling | 9272 |
| | technologies | |
| PPM1K | Abcam | Ab135286 |
| GFP | Clontech | 632375 |
| BDK | Santa Cruz | sc374425 |
| V5 | Genetex | GTX-42525 |
| p-e1a BCKDH ser293 | Abcam | ab200577 |
| e1a BCKDH | Santa Cruz | sc-67200 |
| GAPDH | Sigma | G8795 |
| B-tubulin | Sigma | T5326 |
| ETFA | Abcam | ab110316 |
| COXIV | Li-Cor | 926-42214 |

Bacterial and Virus Strains

| | | |
|---|---|---|
| Ad-CMV-BDK | In house | N/A |
| Ad-CMV-Bgal | In house | N/A |
| Ad-CMV-GFP | Vector Biolabs | 1060 |
| Ad-CMV-PPM1K | Vector Biolabs | ADV-219587 |
| Ad-CMV-ChREBP | In house | N/A |
| Ad-CMV-GFP (control for ChREBP) | In house | N/A |
| Ad-CMV-ΔMTS-BDK | In house | N/A |
| CMV-ΔMTS-BDK-GFP | In house | N/A |

Chemicals, Peptides, and Recombinant Proteins

| | | |
|---|---|---|
| I. BT2, 3,6-Dichlorobenzo[β]thiophene-2- | Sigma | Discontinued - |
| carboxylic acid | | have recently |
| | | validated BT2 |
| | | from Chem-Impex |
| | | Int. INC Cat# |
| | | 25643 |
| GST-tagged human ACL | Sigma | SRP0288 |
| 40 KDa subunit of bovine PKA | Promega | V5161 |

Critical Commercial Assays

| | | |
|---|---|---|
| Rat Insulin ELISA | EMD Millipore | EZRMI-13K |
| V5-tagged protein purification Kit | MBL | 3317 |
| TransIT-293 Transfection Reagent | Mirus | MIR2700 |
| Image-IT Live Mito and Nuclear Labeling Kit | ThermoFisher | I34154 |
| Triglyceride | Abcam | Ab65336 |
| 6-plex Tandem Mass Tag Kit | ThermoFisher | 90061 |
| iScript cDNA synthesis kit | BioRad | 1708890 |
| TRI reagent | MRC | Tr118 |

Deposited Data

| | | |
|---|---|---|
| Raw LC-MS/MS data | ProteomeXchange | TBD |
| | Consortium via | |
| | Pride partner | |
| | repository | |

Experimental Models: Cell Lines

| | | |
|---|---|---|
| Fao hepatoma cells | Sigma | 85061112 |
| Hek293 cells | ATCC | CRL-1573 |

Experimental Models: Organisms/Strains

| | | |
|---|---|---|
| Wistar rats | Charles River | Strain: 003 |
| | Laboratories | |
| Zucker Fatty Rats | Charles River | Zuc-FA/Fa |
| | Laboratories | Strain: 185 |

Oligonucleotides

See Table 2

Recombinant DNA

| | | |
|---|---|---|
| Gateway pDONR223 BDK plasmid | DNASU | HsCd00511364 |
| Gateway pDONR223 ACL plasmid | DNASU | HsCd00399238 |
| pAd/CMV/V5-DEST Bgal plasmid | Thermolife | 49320 |

Software and Algorithms

| | | |
|---|---|---|
| Proteome Discoverer v2.1 | ThermoFisher | |
| PhosphoSitePlus ® | www.phosphosite.org | |
| PRIDE partner repository accession number | ProteomeXchange | PXD009122 |

TABLE 2
Oligonucleotides

| | | |
|---|---|---|
| rPPM1K (taqman) | Life | Rn01410038_m1 |
| hPPM1K (taqman) | Life | Hs00410954_m1 |
| PPIA (taqman) | Life | Rn00690933_m1 |
| rPPM1K | IDT | F- TTTGGGTTCGCAC AGTTGAC (SEQ ID NO: 3) |
| | | R- AAGTCTTTCTCCCGAGGAAGC (SEQ ID NO: 4) |
| rChREBP-a | IDT | F-AGCATCGATCCGACACTCAC (SEQ ID NO: 5) |
| | | R- TGTTCAGCCGAATCTTGTCC (SEQ ID NO: 6) |
| rChREBP-b | IDT | F- AGGT CCCAGGATCCAGTCC (SEQ ID NO: 7) |
| | | R- TGTTCAGCCGAATCTTGTCC (SEQ ID NO: 8) |
| mChREBP | IDT | F- CACTCAGGGAATACACGCCTAC (SEQ ID NO: 9) |
| | | R- ATCTTGGTCTTAGGGTCTTCAGG (SEQ ID NO: 10) |
| hChREBP-b | IDT | F- AGCGGATTCCAGGTGAGG (SEQ ID NO: 11) |
| | | R- TTGTTCAGGCGGATCTTGTC (SEQ ID NO: 12) |
| rBckdk | IDT | F- GTCATCACCATCGCCAATAACG (SEQ ID NO: 13) |
| | | R- TGTGGTGAAGTGGTAGTCCATG (SEQ ID NO: 14) |
| hBckdk | IDT | F- TGAGAAGTGGGTGGACTTTGC (SEQ ID NO: 15) |
| | | R- ATGGCATTCTTGAGCAGCTC (SEQ ID NO: 16) |
| rPklr | IDT | F- TTCCTTCAAGTGCTGTGCAG (SEQ ID NO: 17) |
| | | R- GCAGATCGAGTCACAGCAATG (SEQ ID NO: 18) |
| rFasn | IDT | F- CAAGCAGGCACACACAATGG (SEQ ID NO: 19) |
| | | R- AGTGTTTGTTCCTCGGAGTGAG (SEQ ID NO: 20) |
| rACLY | IDT | F- TTCAAGTATGCCCGGGTTACTC (SEQ ID NO: 21) |
| | | R- TTCCTCGACGTTTGATCAGC (SEQ ID NO: 22) |

Results

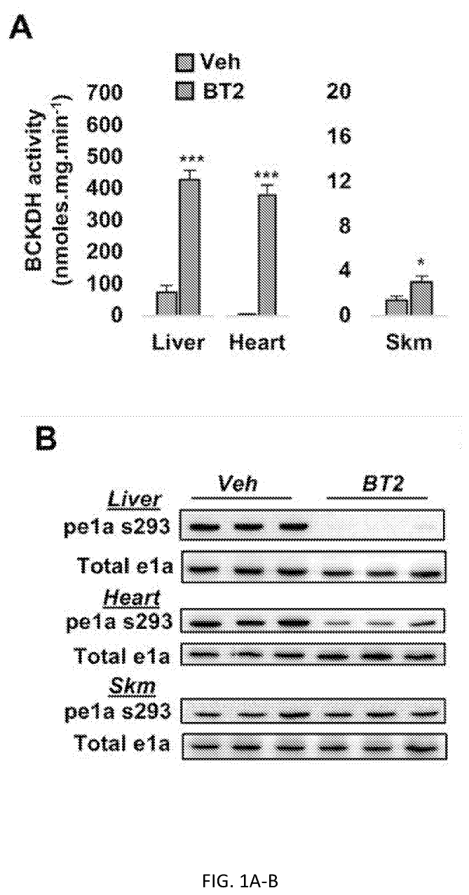

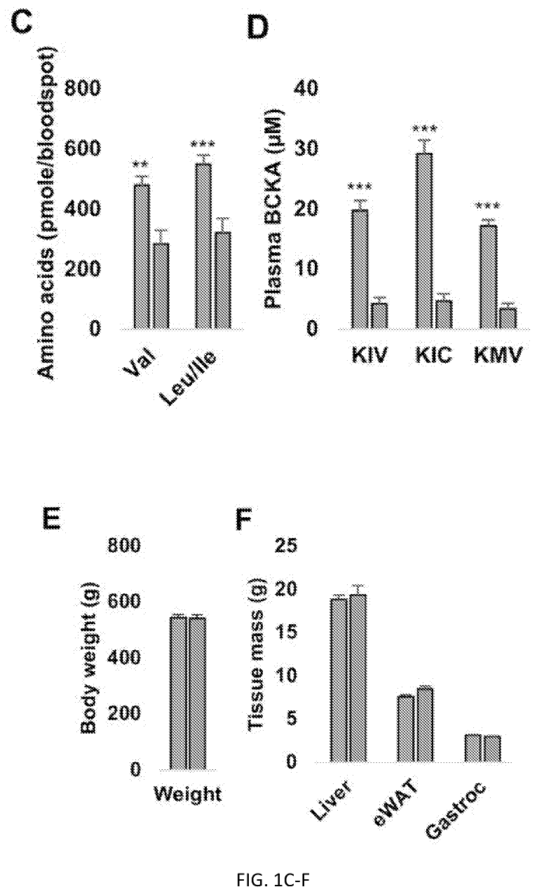

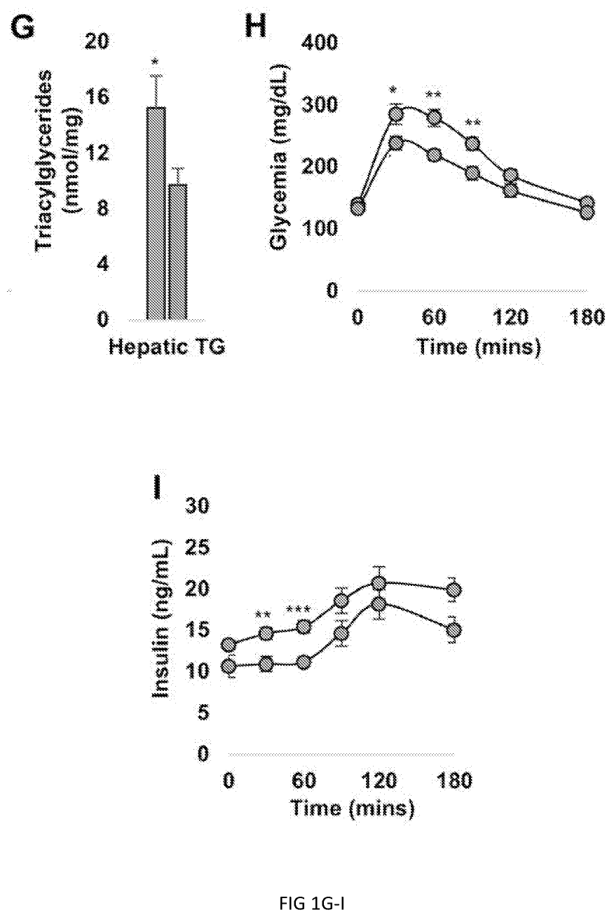

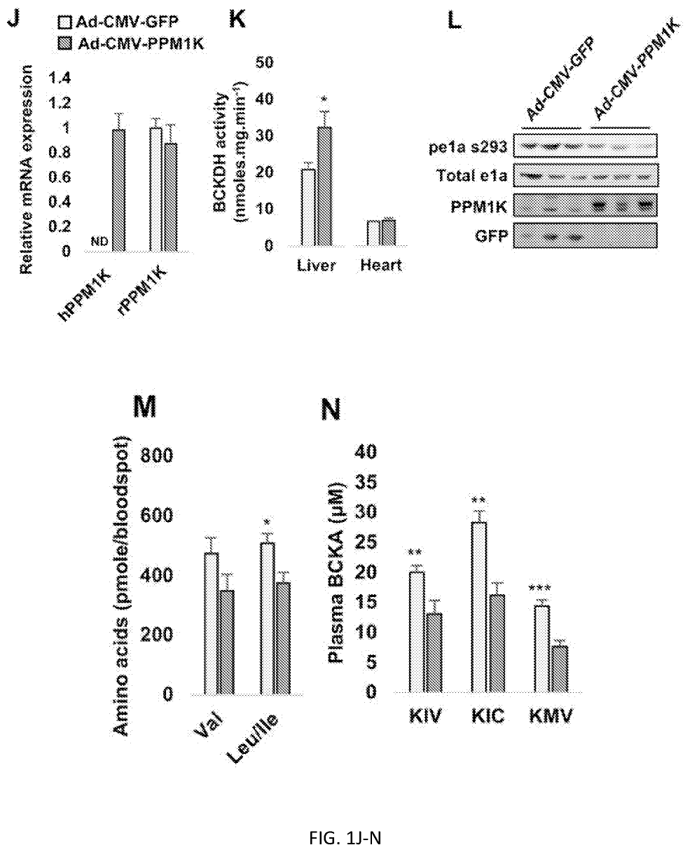

Inhibition of BDK lowers hepatic TG levels and improves glucose tolerance: The potential therapeutic impact of manipulation of the BCKDH complex and its regulatory kinase, BDK, and phosphatase, PPM1K was investigated. Obese and insulin resistant Zucker fatty rats (ZFR) were treated with 3,6-dichlorobenzo(b)thiophene-2-carboxylic acid (BT2), a small molecule inhibitor of BDK (Tso et al., 2014). Daily treatment of ZFR with BT2 (20 mg·kg⁻¹ i.p.) for one week increased BCKDH enzyme activity in liver, heart, and skeletal muscle (FIG. 1A). The increase in BCKDH activity in BT2-treated rats was accompanied by lower levels of BCKDH phosphorylation on serine 293 of the e1a subunit in liver and heart, whereas the small increment in skeletal muscle BCKDH activity was not associated with a detectable change in BCKDH phosphorylation (FIG. 1B). Systemic activation of BCKDH with BT2 lowered circulating BCAA levels, coupled with more dramatic lowering of all three branched-chain α-ketoacids (BCKA), the immediate substrates of BCKDH (FIG. 1C-D). Thus, systemic activation of BCKDH with BT2 is an effective means of lowering circulating BCAA and their α-ketoacids in genetically obese ZFR.

Lowering of BCAA and BCKA via BT2 administration for one week did not affect body, liver, adipose or skeletal muscle weight (FIG. 1E-F). However, BT2-treated ZFR had significantly lower hepatic TG levels (FIG. 1G). Glucose and insulin excursions were also significantly smaller during an intraperitoneal glucose tolerance test (ipGTT) in ZFR treated with BT2 compared to vehicle-treated controls (FIG. 1H-I). Lower glucose excursions accompanied by lower insulin levels reflect improvement in insulin sensitivity in ZFR after a single week of BT2 administration. Thus, inhibition of BDK is an effective approach for correction of abnormalities in glucose, lipid, and amino acid homeostasis in obese animals, even in the absence of weight loss.

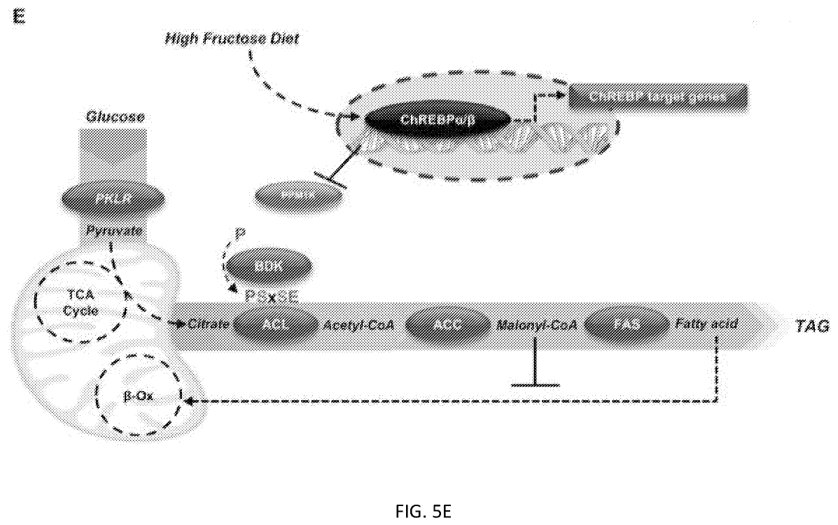

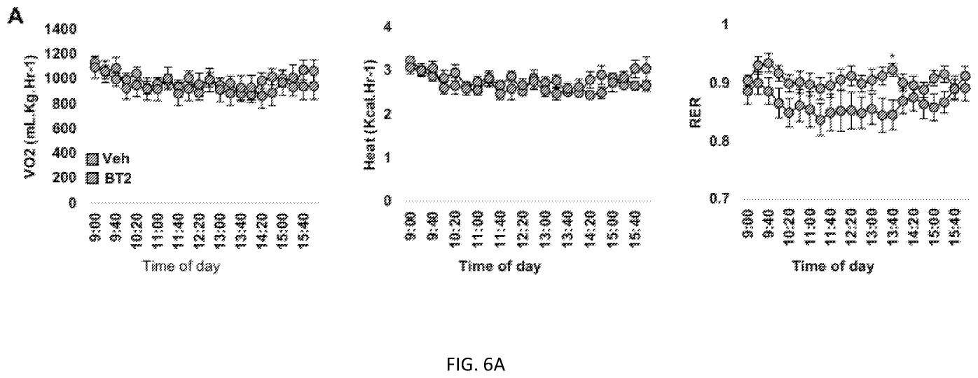

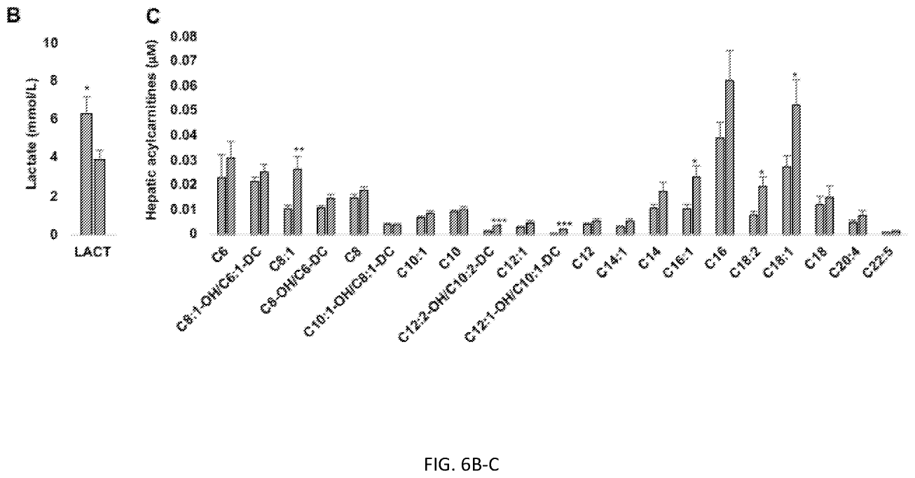

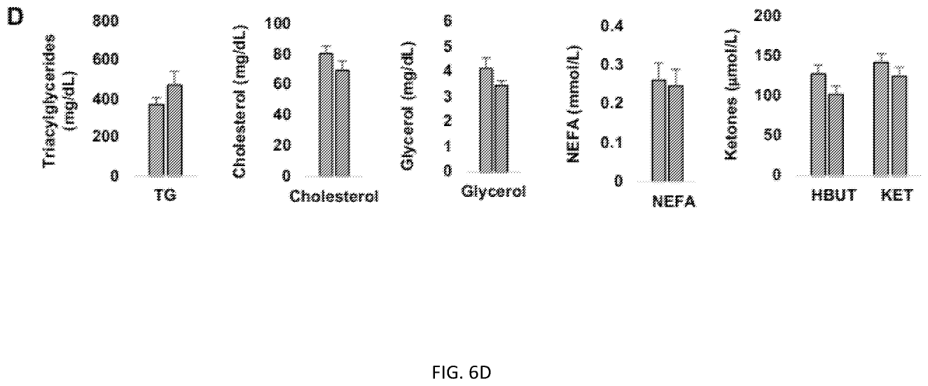

Energy balance was also measured via indirect calorimetry. Treatment of ZFR with BT2 had no impact on 02 consumption or heat production, whereas it lowered the respiratory exchange ratio (RER) in the hours following administration, likely reflecting a shift in substrate preference from glucose to fatty acids (FIG. 6A). Consistent with this interpretation, BT2 treatment resulted in lower levels of lactate in circulation (FIG. 6B). Whereas there were no effects of BT2 treatment on circulating TG, cholesterol, glycerol, non-esterified fatty acids (NEFA), or ketones (FIG. 6D), BT2-treated ZFR exhibited increases in a broad array of even chain acyl-carnitines in liver (FIG. 6C), but not in skeletal muscle. The constellation of elevated even chain acylcarnitines, lower RER, and reduced TG content suggests that inhibition of BDK with BT2 suppresses fat storage and activates fatty acid oxidation in liver.

PPM1K overexpression mirrors the metabolic effects of BDK inhibition: Recombinant adenovirus was used to overexpress human PPM1K as an independent molecular approach for activating BCKDH activity in liver of ZFR. One week after tail-vein administration of recombinant adenoviruses, clear expression of human PPM1K mRNA in liver of Ad-CMV-PPM1K but not Ad-CMV-GFP-treated ZFR was observed (FIG. 1J). Adenovirus-mediated PPM1K overexpression increased hepatic PPM1K protein levels (FIG. 1I), and hepatic but not cardiac BCKDH enzymatic activity (FIG. 1K). As observed with BT2, higher hepatic BCKDH activity in Ad-CMV-PPM1K-treated rats was associated with lower levels of BCKDH phosphorylation on serine 293 compared to Ad-CMV-GFP-treated ZFR (FIG. 1L). Ad-CMV-PPM1K administration also tended to lower valine and significantly lowered leucine/isoleucine levels (FIG. 1M) in concert with a robust and significant lowering of all three BCKA (FIG. 1N).

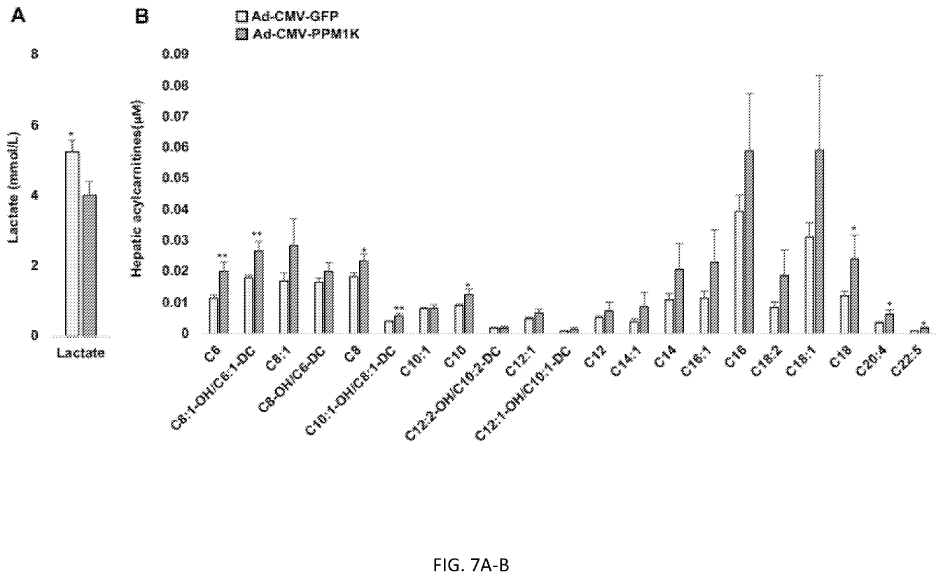

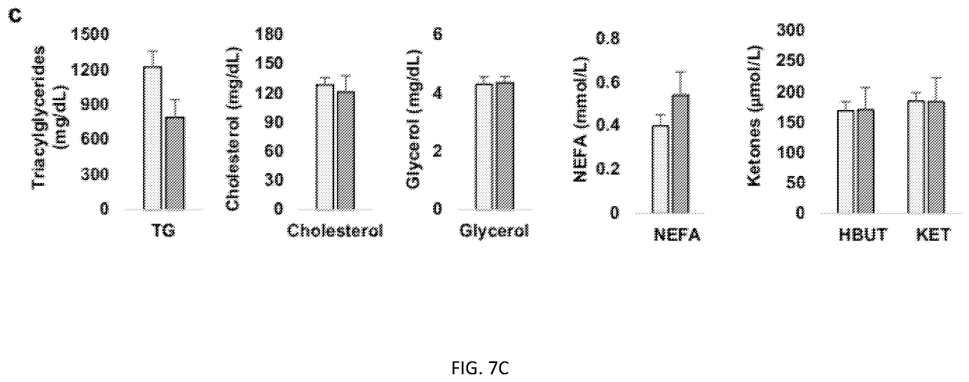

Similar to BT2, Ad-CMV-PPM1K administration had no effect on body, liver, adipose, or skeletal muscle weight over the 7-day study period (FIG. 1O-P), yet significantly lowered hepatic TG content compared to Ad-CMV-GFP-treated ZFR (FIG. 1Q). Like BT2 treatment, administration of Ad-CMV-PPM1K also decreased the glucose excursion during an ipGTT while also tending to lower the insulin excursion (FIG. 1R-S). Again, these effects were accompanied by lower circulating lactate levels (FIG. 7A) and higher levels of even chain acyl-carnitines in liver (FIG. 7B). Also similar to BT2, Ad-CMV-PPM1K treatment had no effects on circulating TG, cholesterol, glycerol, non-esterified fatty acids (NEFA), or ketones (FIG. 7C).

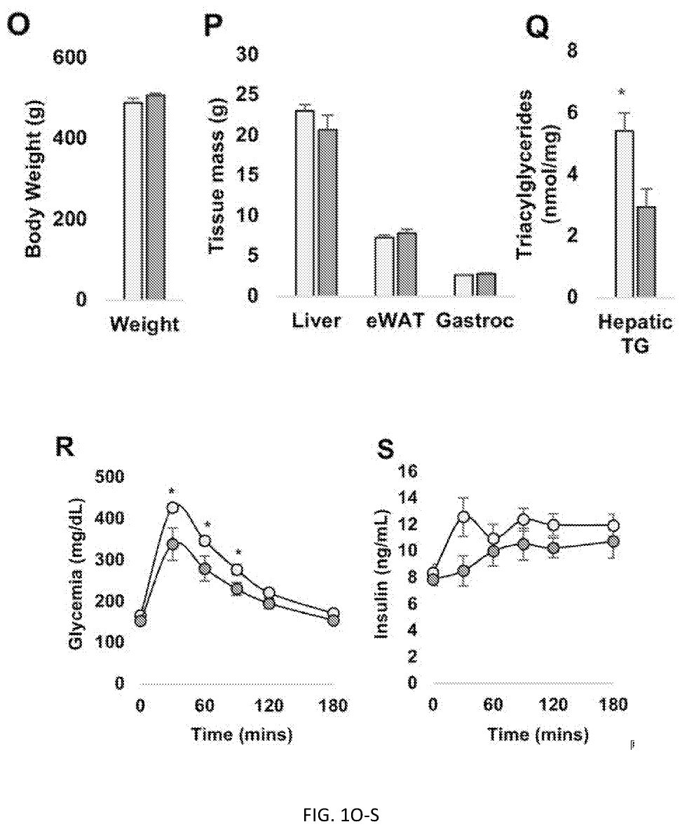

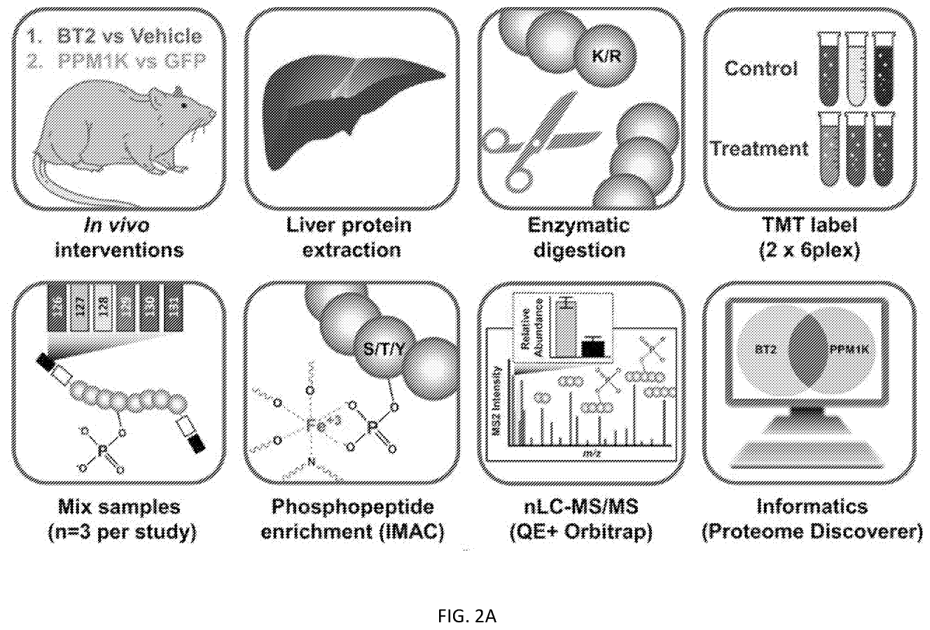

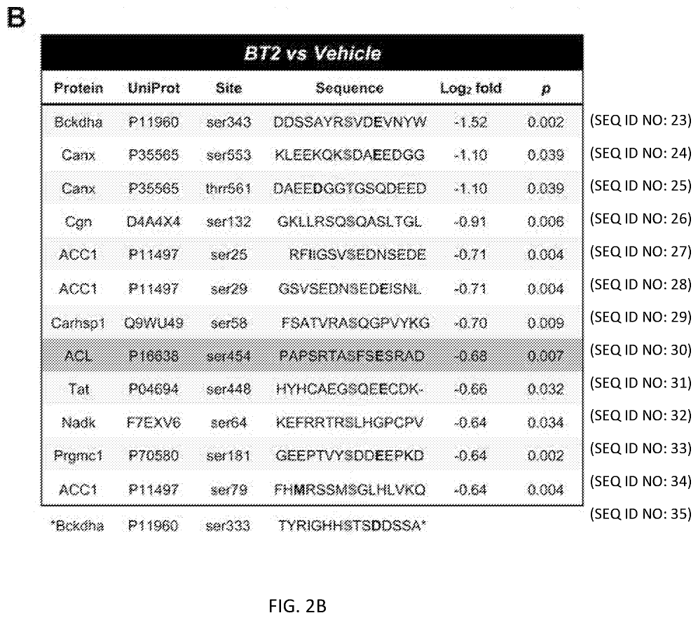

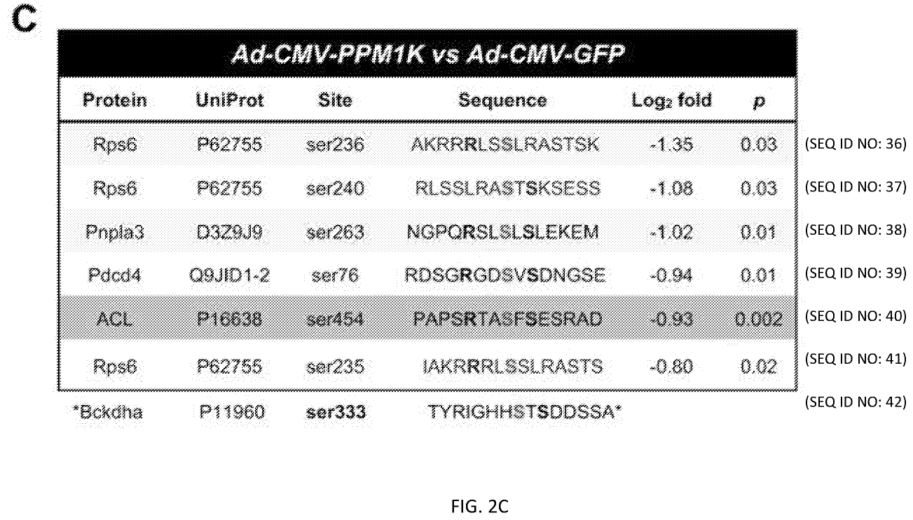

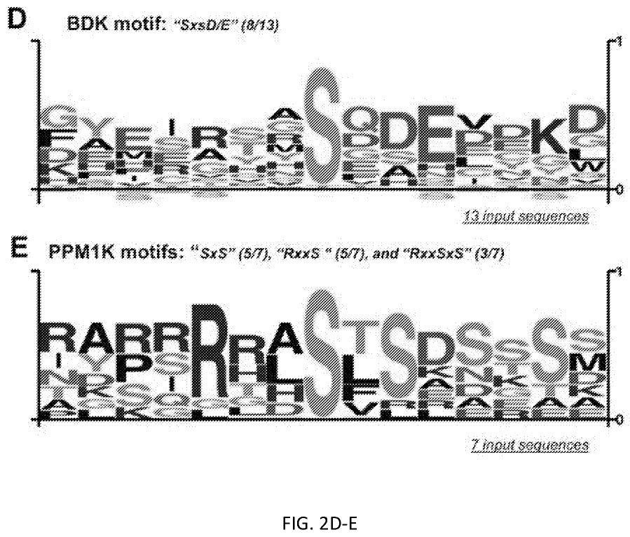

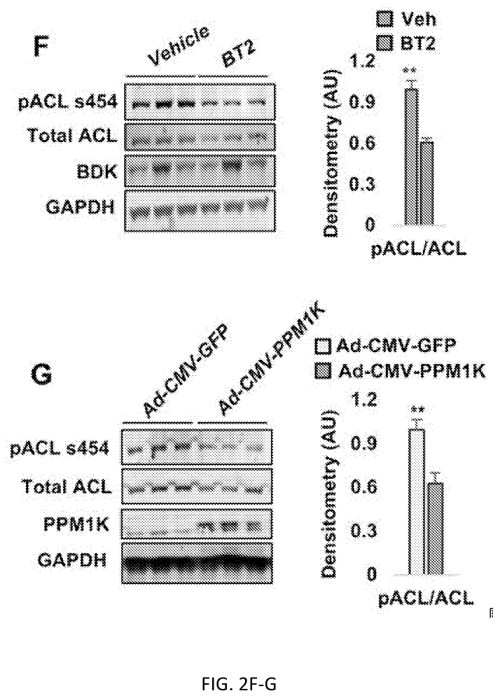

Phospho-proteomics screen reveals substrates in addition to BCKDH for BDK and PPM1K: The broad effects of BT2 and PPM1K overexpression on glucose and lipid metabolism in addition to amino acid metabolism could suggest that BDK and PPM1K have biological substrates in addition to BCKDH. To investigate this idea further, unbiased mass spectrometry-based phospho-proteomics was used to broadly measure site-specific phosphorylation changes in liver samples from both the BT2 study (comparing BT2-treated to vehicle-treated ZFR), and the PPM1K study (comparing Ad-CMV-PPM1K to Ad-CMV-GFP-treated ZFR). A schematic summary of the quantitative phosphoproteomics workflow using peptide labeling with isobaric tags (TMT) and Orbitrap mass spectrometry is shown in FIG. 2A.

5169 phosphopeptides were quantified in livers from the BT2 study and 4350 phosphopeptides in livers from the PPM1K study. Of these, only 11 phosphopeptides encompassing 12 phosphosites from 9 proteins were classified as significantly downregulated in the BT2 study using a threshold of Log 2 fold change ≥−0.585 with P<0.05, whereas 7 phosphopeptides encompassing 6 phosphosites from 4 proteins were classified as significantly downregulated in the PPM1K study (FIG. 2B-C). Serine 454 (serine 455 in humans) of ATP-citrate lyase (ACL) was the only phosphosite found to be significantly downregulated in both studies (FIG. 2B-C). The function of ACL is to cleave citrate to form acetyl CoA and oxaloacetate. Acetyl CoA can then form malonyl CoA, which serves as both the immediate substrate for de novo lipogenesis and an allosteric inhibitor of CPT1 and fatty acid oxidation. The other product of the ACL reaction, oxaloacetate, can be utilized for gluconeogenesis and other metabolic pathways. Phosphorylation of ACL on serine 454 activates ACL and knockout of ACL in genetically obese mice markedly improves glucose tolerance and hepatic steatosis. Thus, a decrease in phosphorylation of ACL in response to BT2 treatment or PPM1K overexpression could contribute to the effects of these interventions on glucose and lipid metabolism described in FIG. 1 and FIGS. 6 and 7.

Serine 293 of BCKDH e1a, identified as serine 333 in proteomics data because of inclusion of the N-terminal mitochondrial targeting sequence, was not observed to be modulated in either the BT2 or PPM1K study, although there was a trend for decreased phosphorylation in the BT2 study (Log₂ fold change of −0.68, p=0.054). Nevertheless, the immunoblot data presented in FIG. 1 clearly demonstrate the expected decrease in phosphorylation of the BCKDH e1a subunit in response to BT2 and Ad-CMV-PPM1K treatment of ZFR. This apparent discrepancy is likely due to a combination of methodological limitations including the tendency of phosphorylation sites immediately after a basic residue to promote missed cleavages by trypsin, complications in detecting multi-site hierarchical phosphorylation within a given peptide, and quantitative interference from peptide co-isolation.

Next, a motif scan was performed to query the sequence similarity around phosphorylated amino acids identified in the proteomics study. Based on the immunoblot data shown in FIG. 1, serine 293 of the BCKDH e1a subunit was included in this analysis. It was hypothesized that BDK and PPM1K substrates would possess common sequence motifs around the phosphosites. Previous work on BCKDH described the sequence “SxxE/D” as required for phosphorylation of BCKDH e1a on ser293 (ser333) and ser303 (ser343) by BDK (Pinna and Ruzzene, 1996). There is no known consensus motif for PPM1K. The flanking sequences for all phosphosites for which phosphorylation was reduced by BT2 treatment or PPM1K overexpression are shown in FIGS. 2B and 2C. It was found that 8/13 input sequences from the BT2 study, including ACL ser454, contained the canonical BDK motif, “SxxE/D” (FIG. 2D). Phosphosite scans of the seven identified phosphosites regulated by PPM1K overexpression revealed two common motifs. All PPM1K-regulated phosphosites contained either an “SxS” (5/7) or an “RxxS” (5/7) motif with three of the seven phosphosites, including ser454 of ACL, possessing both i.e. “RxxSxS” (FIG. 2E). Together these data suggest that PPM1K and BDK likely recognize distinct motifs. Notably, ser293 of BCKDH e1a and ser454 of ACL are surrounded by both the known BDK consensus sequence “SxxD/E” and one or both of the PPM1K motifs (“SxS” or “RxxS”) identified here.

Immunoblot confirmation of regulation of ACL by BDK and PPM1K: Immunoblot analysis was used to generate direct evidence that BDK inhibition and PPM1K overexpression regulate ACL phosphorylation on ser454. Consistent with the phospho-proteomics data, liver lysates from ZFR treated with BT2 or Ad-CMV-PPM1K displayed markedly less ACL phosphorylation, measured with an antibody recognizing phosphorylated ser454 of ACL, compared to samples from ZFR treated with vehicle or Ad-CMV-GFP, respectively (FIG. 2F-G). It was also observed that lower phosphorylation of ACL was associated with a trend for lower abundance of total ACL protein in the BT2 study. Nevertheless, scanning of the immunoblots demonstrated that the reduction in phosphorylation on ser454 remained significant in both the BT2 and PPM1K studies after correction for total ACL abundance (P<0.01). Since acetylation has been reported to stabilize ACL by preventing ubiquitination and subsequent proteasomal degradation, the modest reduction in ACL abundance in the BT2 study may be related to reduced acetyl CoA formation by the dephosphorylated and less active ACL enzyme.

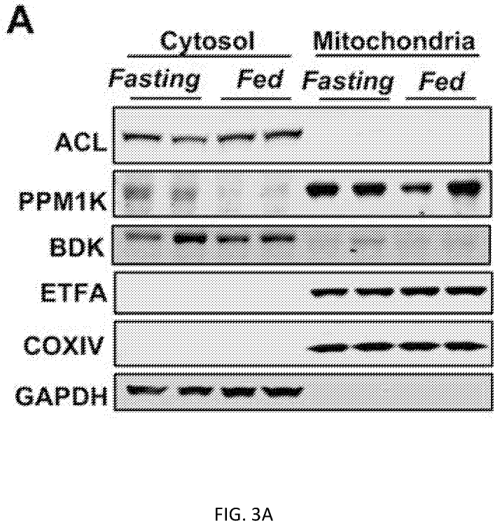

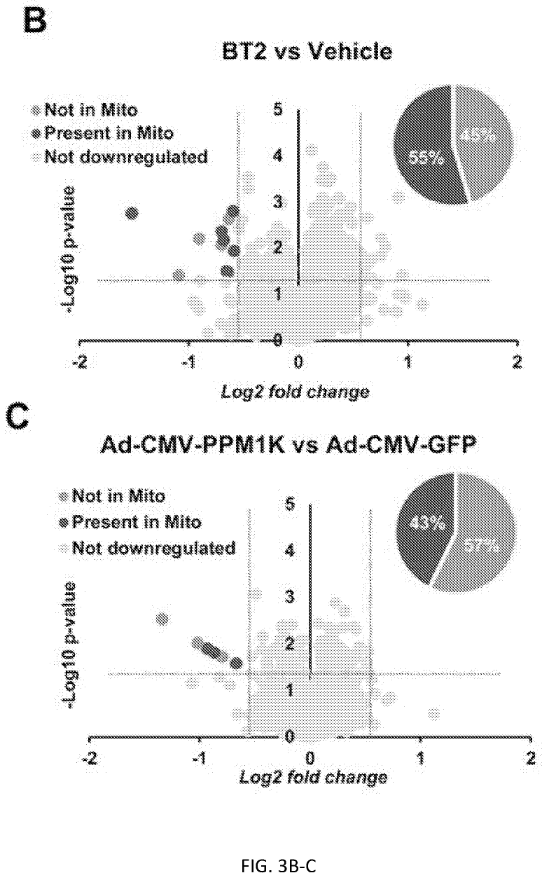

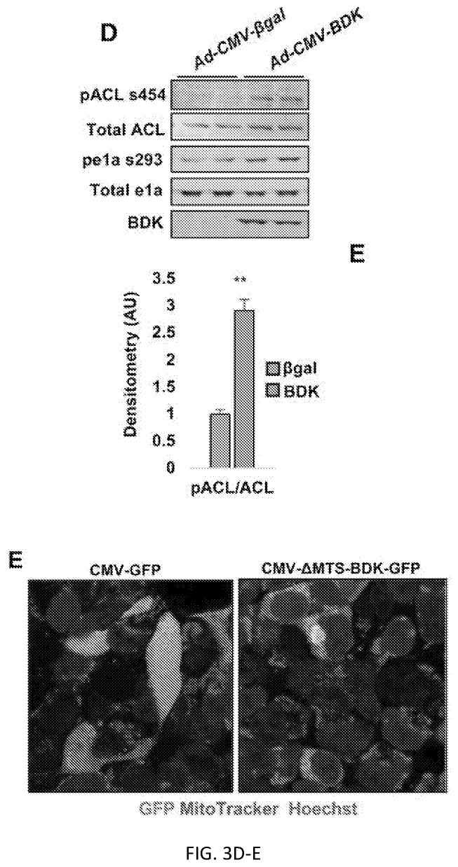

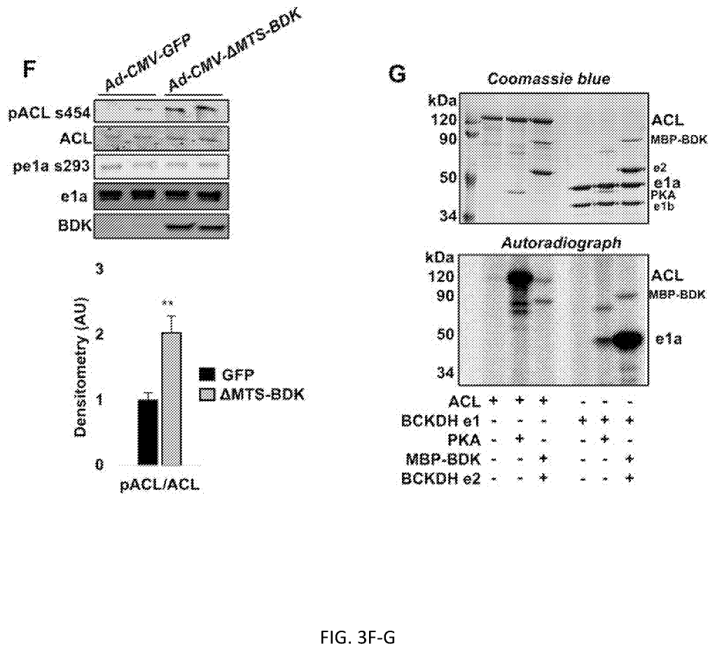

Mitochondrial and cytosolic pools of BDK and PPM1K facilitate regulation of mitochondrial BCKDH and cytosolic ACL: These data suggest that BDK and PPM1K influence the phosphorylation states of ACL and BCKDH, despite the fact that the two target enzymes are known to reside in the cytosolic and mitochondrial subcellular compartments, respectively. In an attempt to resolve this apparent paradox, subcellular fractionation studies of liver extracts taken from lean healthy 8-week old Wistar rats were performed in both fasted and fed states. Cytosolic and mitochondrial fractions were prepared from these samples. Purity was confirmed by blotting for the established mitochondrial markers ETFA and COXIV, and the cytosolic protein GAPDH. The absence of mitochondrial contamination in cytosolic fractions was confirmed by assaying citrate synthase activity. Importantly, ETFA or COXIV protein (FIG. 3A) and citrate synthase activity (not shown) were undetectable in the cytosolic fractions, whereas GAPDH was not detected in the mitochondrial fraction (FIG. 3A). ACL was detected exclusively in the cytosolic fractions, as expected. PPM1K was preferentially found in the mitochondrial fraction, but also clearly detected in the cytosol (FIG. 3A). Surprisingly, BDK was preferentially localized in the cytosolic fraction, but also detected in the mitochondrial fraction. The slightly slower gel migration of cytosolic BDK than its mitochondrial counterpart is consistent with the presence of a previously reported 30-amino acid residue mitochondrial targeting sequence in cytosolic BDK that is cleaved as the enzyme enters the mitochondria. Interestingly, PPM1K protein levels in the cytosol were markedly reduced in the fed compared to fasted states (FIG. 3A). In contrast, cytosolic BDK levels were unaffected by the transition from fasting to feeding (FIG. 3A).

The 11 phosphopeptides identified in the BT2 study and the 7 phosphopeptides from the PPM1K study were also screened against the annotated subcellular localization data for their parent proteins from the Gene Ontology database. Remarkably, 45% (5 of 11) and 57% (4 of 7) of the modified phosphopeptides from the BT2 and PPM1K studies, respectively, are in proteins annotated as extra-mitochondrial (FIGS. 3B-C). Taken together, these studies demonstrate localization of BDK and PPM1K in both the cytosolic and mitochondrial compartments, consistent with their proposed interactions with both the cytosolic ACL enzyme and the mitochondrial BCKDH complex.

Direct phosphorylation of ACL by BDK in an AKT-independent manner: A recombinant adenovirus containing the cDNA encoding BDK (Ad-CMV-BDK) was prepared and used it to express BDK in FAO hepatoma cells in vitro. Treatment of these cells with Ad-CMV-BDK for seventy-two hours increased phosphorylation of ACL on ser454 and the e1a subunit of BCKDH on ser293 (FIG. 3D). These data demonstrate that BDK regulates ACL phosphorylation in a cell autonomous manner independent of hormonal or humoral factors that could have contributed in the in vivo setting.

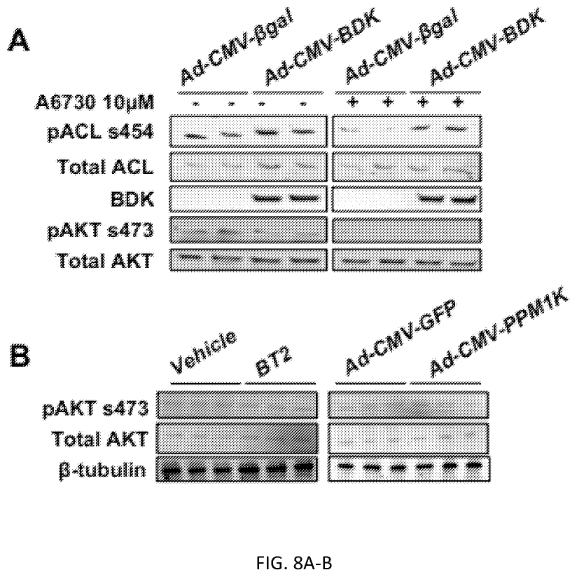

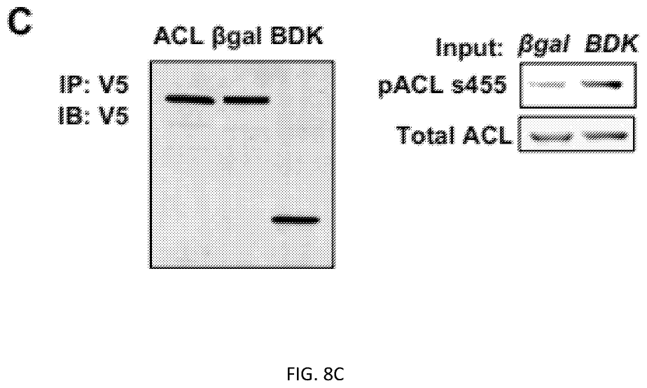

It was next evaluated whether phosphorylation of ACL on ser454 is mediated by AKT. FAO cells were treated with Ad-CMV-BDK or Ad-CMV-βGAL adenoviruses for seventy-two hours, and then incubated in the presence or absence of the pan-AKT inhibitor A6730 (10 μM) for 1 hour. Whereas A6730 had the expected effect to reduce phosphorylation of ser473 on AKT, causing inactivation of the enzyme, the effect of Ad-CMV-BDK to increase ACL phosphorylation on ser454 was readily apparent in the presence of the AKT inhibitor (FIG. 8A). No change in levels of phospho-ser473 AKT in Fao cells treated with Ad-CMV-BDK was observed compared to Ad-CMV-βgal (FIG. 8A), or in the livers of rats treated with BT2 or Ad-CMV-PPM1K compared to their respective controls (FIG. 8B). These findings support the conclusion that BDK-induces phosphorylation of ACL on ser454 independent of AKT activity.

To test if BDK phosphorylates ACL in the cytosolic compartment of living cells in a BCDKH-independent manner, a recombinant adenovirus expressing a form of BDK that lacks its mitochondrial targeting sequence (Ad-CMV-ΔMTS-BDK) was prepared. Using confocal microscopy, it was demonstrated that a GFP tagged ΔMTS-BDK construct is effectively restricted to the cytosolic compartment, and is absent from mitochondria (FIG. 3E). As observed with overexpression of wild type BDK, transfection of FAO cells with Ad-CMV-ΔMTS-BDK for seventy-two hours resulted in increased phosphorylation of ACL on ser454 (FIG. 3F). This occurred absent any change in BCKDH phosphorylation. These studies demonstrate that cytosolic BDK functions to increase ACL phosphorylation in a manner that is independent from its effect on its canonical mitochondrial target BCKDH.

To determine if BDK can phosphorylate ACL directly, a maltose-binding protein (MBP)-tagged version of mature BDK that lacks the mitochondrial-targeting pre-sequence (MBP-BDK) was expressed and purified and mixed with purified ACL in the presence of [³²P] ATP. ACL phosphorylation by protein kinase A (PKA) was evaluated with purified ACL and purified PKA. The e2 component of the BCKDH complex, which facilitates phosphorylation of BCKDH by BDK, was also included in the reaction mixture for experiments involving BDK. In contrast, the catalytic subunit of PKA neither interacts with nor is activated by BDKDH e2; therefore, BCKDH e2 was not included in lanes where PKA activity was studied. PKA caused a clear increase in ³²P labeling of ACL. MBP-BDK also caused phosphorylation of ACL, albeit to a lesser extent than PKA (FIG. 3G). MBP-BDK also caused a robust increase in phosphorylation of the purified e1a subunit of BCKDH. Intriguingly, PKA caused a lesser, but still clear increase in BCKDH phosphorylation (FIG. 3G). In a parallel experiment, purified V5-tagged BDK and ACL proteins were mixed, and ACL phosphorylation was measured by immunoblot analysis. This study confirmed that BDK specifically phosphorylates ser455 of the V5-tagged human ACL (corresponding to ser454 in rats; FIG. 8C).

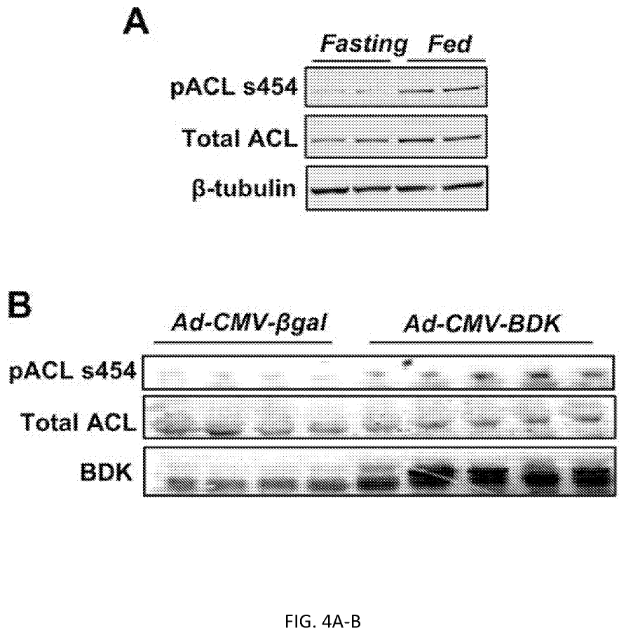

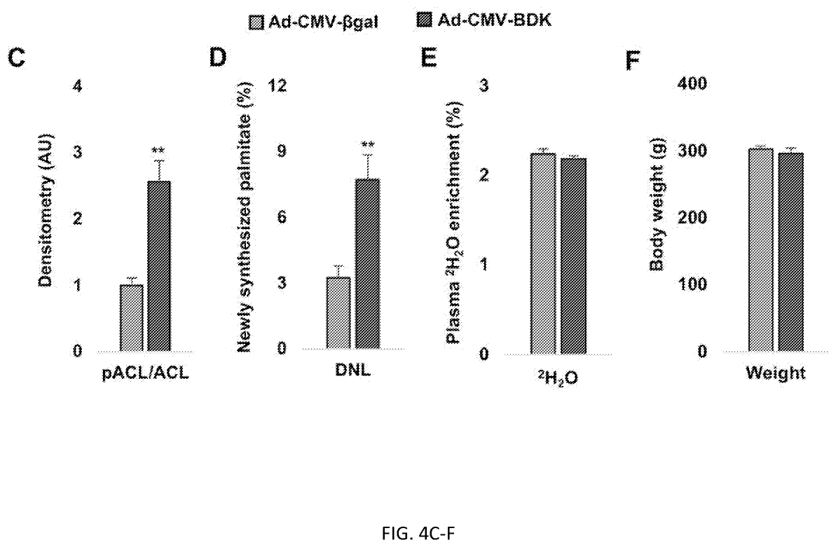

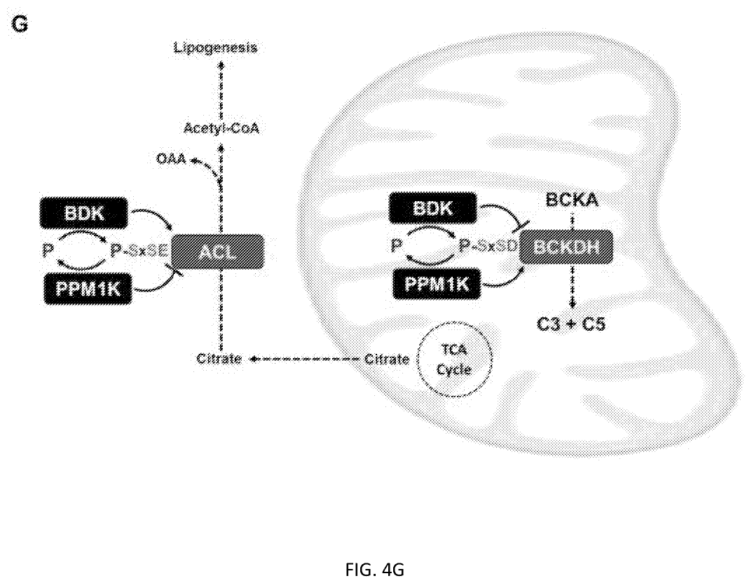

BDK stimulates ACL phosphorylation and de novo lipogenesis in vivo: In line with a physiologically relevant role for ACL phosphorylation, phosphorylation on ser454 is higher in liver samples from ad-lib fed compared to fasted rats (FIG. 4A), a state where glucose is abundant and flux through ACL is increased to provide malonyl CoA for lipogenesis and to curtail fatty acid oxidation. Notably, the increase in ACL phosphorylation on ser454 in the fed state corresponds to the decrease in PPM1K protein abundance observed in the cytosolic fraction of livers from fed rats (FIG. 3A).

To test the direct effects of modulation of the BDK:PPM1K ratio in vivo, the Ad-CMV-BDK adenovirus, which encodes full-length BDK inclusive of its MTS, or the Ad-CMV-□GAL control virus, was injected into lean healthy ad-lib fed Wistar rats via tail-vein injection. To measure de novo lipogenesis, a bolus of ²H₂O was delivered and then p ²H₂O was provided in the drinking water for two days prior to sacrifice. At sacrifice, animals treated with Ad-CMV-BDK had clear increases in liver BDK protein levels compared to Ad-CMV-□GAL-treated controls (FIG. 4B). Overexpression of BDK increased the levels of ACL phosphorylation on ser454 (FIGS. 4B and 4C) and this occurred concomitant with a 2.4-fold increase in deuterium labeling of palmitate in liver of Ad-CMV-BDK compared to Ad-CMV-□Gal-treated rats (FIG. 4D; p<0.001). The Ad-CMV-BDK and Ad-CMV-□GAL-treated groups had the same level of steady-state ²H₂O enrichment in plasma and identical body weights (FIGS. 4E and 4F). Thus hepatic expression of BDK, a kinase previously known only as a regulator of BCKDH and BCAA metabolism, is sufficient to increase phosphorylation of a critical lipogenic enzyme and activate de novo lipogenesis (FIG. 4G).

ChREBP Regulates Hepatic BDK and PPM1K Expression as a Component of a Lipogenic Transcriptional Program: The transcription factor Carbohydrate-Response Element Binding Protein (ChREBP, also known as Mlxipl) responds to cellular hexose phosphate levels to coordinate expression of multiple glycolytic and lipogenic genes, including acetyl CoA carboxylase (ACC), fatty acid synthase (Fasn), the liver isoform of pyruvate kinase (Pklr) and ACL. Given the role demonstrated herein of BDK and PPM1K in regulation of ACL phosphorylation and lipogenesis, the possibility that these genes are regulated by ChREBP as part of a “lipogenic gene cluster” was next evaluated. Genomic sequences across a broad array of species were searched, and it was found that an enhancer upstream of the BDK gene containing this regulatory motif is conserved in humans, non-human primates, and a wide range of mammals including rats, but is surprisingly absent in mice (FIG. 5A). Expression of the ChREBP-β isoform is an excellent marker of cellular ChREBP activity. In liver biopsy samples from 86 overnight fasted human subjects with non-alcoholic fatty liver disease (NAFLD) (49 male, 37 female, age range 18-83; median age 52), expression of ChREBP-β has been demonstrate to correlate with expression of Pklr and Fasn (Kim et al., 2016). Accordingly, the association of ChREBP-β and BDK transcript levels in these same human samples was evaluated. A similar correlation is demonstrated as observed for the classical ChREBP target genes (R²=0.34, p<0.001; FIG. 5B). Lean rats were then fasted and refed with either standard chow or a high-fructose diet to activate hepatic ChREBP-β (Kim et al., 2016). ChREBP-β mRNA levels increased 40-fold in response to high-fructose and was accompanied by marked increases in Fasn, Pklr, and ACL, as well as BDK transcript levels (FIG. 5C). In contrast, PPM1K mRNA levels were suppressed by 35% in response to high-fructose refeeding.

To specifically assess the role of ChREBP, a recombinant adenovirus containing the cDNA encoding mouse ChREBP-β (Ad-CMV-mChREBP-β) was constructed. Ad-CMV-mChREBP-β or an Ad-CMV-GFP control virus was injected into 10 week-old Wistar rats by tail vein injection. Seven days after adenovirus administration, rats that received Ad-CMV-mChREBP-β exhibited increased hepatic expression of mChREBP-β compared to Ad-CMV-GFP-treated rats (P<0.01, FIG. 5D). Overexpression of ChREBP-β mimicked the effect of fructose refeeding by increasing BDK and reducing PPM1K transcript levels compared to Ad-CMV-GFP control rats (P<0.01, FIG. 5D).

Discussion: It is demonstrated herein that BDK and PPM1K, the kinase and phosphatase pair that control BCKDH activity and BCAA levels, also modulate hepatic lipid metabolism by regulating reversible phosphorylation of ATP citrate lyase (ACL) on ser454 (FIG. 4G). ACL is an important enzyme in de novo lipogenesis and regulation of fatty acid oxidation due to its contributions to production of cytosolic acetyl CoA and malonyl CoA from citrate. In contrast to phosphorylation of BCKDH e1a on ser293, which results in inhibition of enzyme activity, phosphorylation of ACL on ser454 is activating, leading to increased generation of acetyl-CoA and malonyl CoA, the latter serving as the immediate substrate for lipogenesis. Increased malonyl CoA levels also inhibit fatty acid oxidation via allosteric inhibition of carnitine palmitoyltransferase-1. Consistent with this construct, it is demonstrated herein that modulation of the ratio of BDK:PPM1K activities in favor of PPM1K by two distinct experimental approaches not only activates BCKDH to lower BCAA and BCKA levels, but also results in marked reduction in hepatic steatosis, lowering of RER, and increased hepatic even-chain acylcarnitines, all consistent with reduced lipogenesis and increased fatty acid oxidation in the liver. It was also observed improved glucose tolerance in response to those maneuvers, possibly secondary to the marked lowering of hepatic triglyceride content. These improvements in metabolic health suggest that the BDK:PPM1K axis serves as a metabolic regulatory node that integrates BCAA, glucose, and lipid metabolism via two distinct phosphoprotein targets.

These unanticipated findings led to several important questions: 1) How can BCKDH e1a and ACL be BDK and PPM1K substrates, when one of the target enzymes (BCKDH) resides in the mitochondrial matrix, whereas the other (ACL) clearly has a cytosolic localization? 2) Are PPM1K, BDK, and phosphorylation of ACL coordinately regulated in response to fasting and refeeding? 3) Is BDK capable of direct phosphorylation of ACL?

With regard to the first question, subcellular fractionation studies revealed that BDK and PPM1K are clearly detectable in both the mitochondrial and cytosolic subcellular fractions, thus making it possible for these enzymes to interact with both the BCKDH and ACL substrates. The preferential presence of BDK in the cytosol is consistent with the rather low copy number of BDK bound to the 24-mer transacylase (E2) core of mitochondrial BCKDH from rat liver. It was also shown that a BDK variant lacking its mitochondrial targeting sequence is expressed in the cytosol, where it phosphorylates ACL in a BCKDH-independent manner. As to the second question, phosphorylation of ser454 on ACL is clearly increased in the fasted to fed transition. Interestingly, this increase is accompanied by a decline in the level of PPM1K protein in the cytosolic, but not the mitochondrial compartment. Concerning the third question, analysis of the amino acid sequence of ACL and comparison to other peptides identified in the phosphoproteomics screen described herein suggests that it could be directly regulated by both BDK and PPM1K due to the presence of a dual BDK-PPM1K motif surrounding the regulatory phosphosite. Moreover, studies summarized in FIG. 3 with purified proteins demonstrate direct phosphorylation of ACL on ser454 by BDK. Collectively, these data provide support for a previously unappreciated role for BDK and PPM1K in the regulation of hepatic lipid metabolism.

The literature concerning ACL regulation is scattered over the past twenty years, and in many ways does not present a coherent picture. Moreover, the physiologic significance (or lack thereof) of multiple mechanisms for regulation of ACL has never been fully explored. For example, it is very unlikely that PKA, an enzyme activated by glucagon and other catabolic effectors associated with the fasted state, would play a physiologic role in increasing hepatic ACL phosphorylation and activity in anabolic, fed conditions. On the other hand, the increase in ACL phosphorylation that occurs in the transition from the fasted to the fed state could reasonably be mediated by insulin signaling through the Akt pathway. The findings described herein demonstrate that modulation of the BDK/PPM1K ratio affects ACL phosphorylation in an Akt-independent fashion, both in isolated cells, and in liver of living animals. Importantly, just as Akt can be activated by insulin in the anabolic state, herein it is shown that levels of cytosolic PPM1K protein decrease in response to feeding, consistent with a physiological role of this new mechanism.

Obesity is a setting in which “selective insulin resistance” appears, a scenario where insulin fails to suppress hepatic glucose output but continues to promote lipogenesis. Increases in the hepatic BDK:PPM1K ratio may cause ACL to be constitutively phosphorylated, such that it no longer responds to fasting in the manner demonstrated in lean rats (FIG. 4A). This model also aligns with findings linking the global metabolic transcription factor ChREBP with expression of BDK and PPM1K. The ChREBP-β isoform is a particularly potent activator of lipogenesis in liver that is induced by excess consumption of sucrose as found in soft drinks and other sugar-containing foods common in western diets. It is possible that overnutrition, particularly when involving diets high in fructose, leads to activation of ChREBP in the liver, which drives increased expression of genes encoding classical enzymes of de novo lipogenesis (DNL), including PKLR, ACL, ACC, and FAS. It is further possible that upregulation of BDK and downregulation of PPM1K by ChREBP stimulates the DNL pathway by phosphorylation and activation of ACL, thus adding BDK and PPM1K to the panel of genes regulated by ChREBP to enhance fatty acid synthesis and development of dyslipidemia (FIG. 5E). Simultaneously, the increased BDK:PPM1K ratio leads to increased phosphorylation and inhibition of BCKDH, contributing to the obesity-linked rise in circulating BCAA and BCKA. These findings suggest that BDK and PPM1K represent a previously unidentified class of ChREBP-β regulated, lipogenesis-activating genes that perform their function via post-translational modulation of a key enzyme activity (ACL) rather than by playing a direct catalytic role in the metabolic conversion of glucose to lipids.

In addition to drawing attention to serine 454 of ACL as a phosphosite that is regulated by both BDK and PPM1K, the phospho-proteomics screen described herein identified several additional sites in other proteins. For example, serine 25, serine 29, and serine79 of the lipogenic enzyme acetyl-coA carboxylase 1 (ACC1) were found to be less phosphorylated in BT2-treated compared to vehicle-treated ZFR. Serine 25 and 29 are known to be phosphorylated in response to insulin, when ACC1 activity is high, whereas serine 79 is the highly studied 5′ AMP-activated protein kinase (AMPK) regulatory site that inhibits ACC1 activity. While these data suggest that BT2 might mediate some of its effects through regulation of ACC1 phosphorylation and activity, the net effect of these multiple changes in phosphorylation on enzyme activity remains to be determined for ACC1, as well as the other candidate phosphoproteins listed in FIGS. 2B and 2C.

In conclusion, the findings described herein shed new light on mechanisms underlying the strong relationship between elevated BCAA and cardiometabolic diseases by showing that the BDK/PPM1K kinase/phosphatase pair regulate both BCAA and lipid metabolism. The potential translational significance of the present work is further highlighted by the finding that manipulation of the BDK:PPM1K ratio to favor PPM1K via BT2 treatment or PPM1K overexpression lowers liver TG levels and blood glucose excursions in highly obese and insulin resistant ZFR. Thus, this study introduces regulation of ACL by BDK and PPM1K as part of a regulatory node, that when modulated, contributes to simultaneous improvements in lipid, glucose and amino acid metabolism, even in the absence of weight loss.

### REFERENCES

Davie, J. R., Wynn, R. M., Meng, M., Huang, Y. S., Aalund, G., Chuang, D. T., and Lau, K. S. (1995). Expression and characterization of branched-chain alpha-ketoacid dehydrogenase kinase from the rat. Is it a histidine-protein kinase? J. Biol. Chem. 270, 19861-19867.
Ferrara, C. T., Wang, P., Neto, E. C., Stevens, R. D., Bain, J. R., Wenner, B. R., Ilkayeva, O. R., Keller, M. P., Blasiole, D. A., Kendziorski, C., et al. (2008). Genetic networks of liver metabolism revealed by integration of metabolic and transcriptional profiling. PLoS Genet. 4, e1000034.
Glynn, E. L., Piner, L. W., Huffman, K. M., Slentz, C. A., Elliot-Penry, L., AbouAssi, H., White, P. J., Bain, J. R., Muehlbauer, M. J., Ilkayeva, O. R., et al. (2015). Impact of combined resistance and aerobic exercise training on branched-chain amino acid turnover, glycine metabolism and insulin sensitivity in overweight humans. Diabetologia 58, 2324-2335.
Hornbeck, P. V., Zhang, B., Murray, B., Kornhauser, J. M., Latham, V., and Skrzypek, E. (2015). PhosphoSitePlus, 2014: mutations, PTMs and recalibrations. Nucleic Acids Res. 43, D512-D520.
Jensen, M. V., Haldeman, J. M., Zhang, H., Lu, D., Huising, M. O., Vale, W. W., Hohmeier, H. E., Rosenberg, P., and Newgard, C. B. (2013). Control of Voltage-gated Potassium Channel Kv2.2 Expression by Pyruvate-Isocitrate Cycling Regulates Glucose-stimulated Insulin Secretion. J. Biol. Chem. 288, 23128-23140.
Kim, M.-S., Krawczyk, S. A., Doridot, L., Fowler, A. J., Wang, J. X., Trauger, S. A., Noh, H.-L., Kang, H. J., Meissen, J. K., Blatnik, M., et al. (2016). ChREBP regulates fructose-induced glucose production independently of insulin signaling. J. Clin. Invest. 126, 4372-4386.
Lavine, J. A., Raess, P. W., Davis, D. B., Rabaglia, M. E., Presley, B. K., Keller, M. P., Beinfeld, M. C., Kopin, A. S., Newgard, C. B., and Attie, A. D. (2010). Contamination with E1A-positive wild-type adenovirus accounts for species-specific stimulation of islet cell proliferation by CCK: a cautionary note. Mol. Endocrinol. 24, 464-467.
Newgard, C. B., An, J., Bain, J. R., Muehlbauer, M. J., Stevens, R. D., Lien, L. F., Haqq, A. M., Shah, S. H., Arlotto, M., Slentz, C. A., et al. (2009). A branched-chain amino acid-related metabolic signature that differentiates obese and lean humans and contributes to insulin resistance. Cell Metab. 9, 311-326.
Pinna, L. A., and Ruzzene, M. (1996). How do protein kinases recognize their substrates? Biochim. Biophys. Acta 1314, 191-225.
Ronnebaum, S. M., Ilkayeva, O., Burgess, S. C., Joseph, J. W., Lu, D., Stevens, R. D., Becker, T. C., Sherry, A. D., Newgard, C. B., and Jensen, M. V (2006). A pyruvate cycling pathway involving cytosolic NADP-dependent isocitrate dehydrogenase regulates glucose-stimulated insulin secretion. J. Biol. Chem. 281, 30593-30602.
Seiler, C. Y., Park, J. G., Sharma, A., Hunter, P., Surapaneni, P., Sedillo, C., Field, J., Algar, R., Price, A., Steel, J., et al. (2014). DNASU plasmid and PSI: Biology-Materials repositories: resources to accelerate biological research. Nucleic Acids Res. 42, D1253-D1260.
Tomcik, K., Ibarra, R. A., Sadhukhan, S., Han, Y., Tochtrop, G. P., and Zhang, G.-F. (2011). Isotopomer enrichment assay for very short chain fatty acids and its metabolic applications. Anal. Biochem. 410, 110-117.
Tso, S.-C., Gui, W.-J., Wu, C.-Y., Chuang, J. L., Qi, X., Skvora, K. J., Dork, K., Wallace, A. L., Morlock, L. K., Lee, B. H., et al. (2014). Benzothiophene carboxylate derivatives as novel allosteric inhibitors of branched-chain α-ketoacid dehydrogenase kinase. J. Biol. Chem. 289, 20583-20593.
White, P. J., Lapworth, A. L., An, J., Wang, L., McGarrah, R. W., Stevens, R. D., Ilkayeva, O., George, T., Muehlbauer, M. J., Bain, J. R., et al. (2016). Branched-chain amino acid restriction in Zucker-fatty rats improves muscle insulin sensitivity by enhancing efficiency of fatty acid oxidation and acyl-glycine export. Mol. Metab. 5, 538-551.

All references, including publications, patent applications, and patents, cited herein are hereby incorporated by reference to the same extent as if each reference were individually and specifically indicated to be incorporated by reference and were set forth in its entirety herein.

Preferred embodiments of this invention are described herein, including the best mode known to the inventors for carrying out the invention. Variations of those preferred embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

1. A method for treating metabolic disease in a subject in need thereof, comprising administering to the subject a therapeutically effective amount of one or more branched-chain ketoacid dehydrogenase complex (BCKDH) agonists.

2. The method of claim 1, wherein the one or more BCDHK agonists are selected from BDK kinase inhibitors, PPM1K agonists, and combinations thereof.

3. The method of claim 1 or 2, wherein the one or more BCDHK agonists are selected from small molecules, antibodies, aptamers, nucleic acids, and proteins.

4. The method of any one of claims 1-3, wherein the one or more BCDHK agonists comprise one or more benzothiophene carboxylate derivatives.

5. The method of claim 4, wherein the one or more benzothiophene carboxylate derivatives are selected from (S)-α-cholorophenylproprionate ((S)-CPP)), (N-(4-amino-1,2,5-oxadiazol-3-yl)-3,6-dichlorobenzo[b]thiophene-2-carboxamide) (BT1), (3,6-dichlorobenzo[b]thiophene-2-carboxylic acid) (BT2), (3-chloro-6-fluorobenzo[b]thiophene-2-carboxylic acid) (BT2F), and (N-(4-acetamido-1,2,5-oxadiazol-3-yl)-3,6-dichlorobenzo[b]thiophene-2-carboxamide) (BT3).

6. The method of claim 5, wherein the benzothiophene carboxylate derivative comprises BT2.

7. The method of any one of claims 1-6, wherein the metabolic disease is selected from obesity, insulin-resistance, diabetes, metabolic syndrome, alcoholic steatohepatitis, and NAFLD.

8. The method of claim 7, wherein the metabolic disease is NAFLD.

9. A method of treating NAFLD in a subject, comprising administering to the subject a therapeutically effective amount of (3,6-dichlorobenzo[b]thiophene-2-carboxylic acid) (BT2).

10. The method of claim 8 or 9, wherein the NAFLD is non-alcoholic steatohepatitis.

11. The method of any one of claims 1-10, wherein the subject is a human.

12. The method of any one of claims 1-11, wherein the subject is overweight or obese.

13. The method of any one of claims 1-12, wherein the subject is female.

14. The method of any one of claims 1-13, wherein the subject expresses the Ile148Met variant of PNPLA3.

15. The method of any one of claims 1-14, further comprising performing surgery on the subject.

16. The method of claim 15, wherein the surgery comprises bariatric surgery.

17. A composition comprising one or more branched-chain ketoacid dehydrogenase complex (BCKDH) agonists for use in a method of treating metabolic disease in a subject.

18. The composition of claim 17, wherein the one or more BCDHK agonists are selected from BDK kinase inhibitors, PPM1K agonists, and combinations thereof.

19. The composition of claim 17 or 18, wherein the one or more BCDHK agonists are selected from small molecules, antibodies, aptamers, nucleic acids, and proteins.

20. The composition of any one of claims 17-19, wherein the one or more BCDHK agonists comprise one or more benzothiophene carboxylate derivatives.

21. The composition of claim 20, wherein the one or more benzothiophene carboxylate derivatives are selected from (S)-α-cholorophenylproprionate ((S)-CPP)), (N-(4-amino-1,2,5-oxadiazol-3-yl)-3,6-dichlorobenzo[b]thiophene-2-carboxamide) (BT1), (3,6-dichlorobenzo[b]thiophene-2-carboxylic acid) (BT2), (3-chloro-6-fluorobenzo[b]thiophene-2-carboxylic acid) (BT2F), and (N-(4-acetamido-1,2,5-oxadiazol-3-yl)-3,6-dichlorobenzo[b]thiophene-2-carboxamide) (BT3).

22. The composition of claim 21, wherein the benzothiophene carboxylate derivative comprises BT2.

23. The composition of any one of claims 17-22, wherein the metabolic disease is selected from obesity, insulin-resistance, diabetes, metabolic syndrome, alcoholic steatohepatitis, and NAFLD.

24. The composition of claim 23, wherein the metabolic disease is NAFLD.

25. A composition comprising (3,6-dichlorobenzo[b]thiophene-2-carboxylic acid) (BT2) for use in a method of treating NAFLD in a subject.

26. The composition of any one of claims 17-25, wherein the NAFLD is non-alcoholic steatohepatitis.

27. The composition of any one of claims 17-26, wherein the subject is a human.

28. The composition of any one of claims 17-27, wherein the subject is overweight or obese.

29. The composition of any one of claims 17-28, wherein the subject is female.

30. The composition of any one of claims 17-29, wherein the subject expresses the Ile148Met variant of PNPLA3.
